(19) United States
(12) Patent Application Publication
(10) Pub. No.: US 2007/0238184 A1
(43) Pub. Date: Oct. 11, 2007

(54) Amyloid beta protein channel structure and uses thereof in identifying potential drug molecules for neurodegenerative diseases

(75) Inventors: Ratnesh Lal, Goleta, CA (US); Arjan Quist, Lombard, IL (US); Sungho Jin, San Diego, CA (US); Hai Lin, Goleta, CA (US)

Correspondence Address: BEYER WEAVER LLP, P.O. BOX 70250, OAKLAND, CA, 94612-0250, US

(73) Assignee: The Regents of the University of California

(21) Appl. No.: 11/450,146

(22) Filed: Jun. 9, 2006

Related U.S. Application Data
(60) Provisional application No. 60/692,048, filed on Jun. 16, 2005.

(51) Int. Cl. G01N 33/00; B01J 47/00; C01B 31/00

(52) U.S. Cl. 436/86; 422/255; 423/445 B

(57) ABSTRACT

The present invention relates to a novel channel structure of human amyloid beta protein (AbP) in lipid membranes and a rapid, quantitative and specific assay for screening test compounds, such as drugs, ligands (natural or synthetic), proteins, peptides and small organic molecules for their ability to bind and block the membrane AbP channels. The invention further relates to screening and identifying therapeutically relevant compounds for treating Alzheimer's disease and other disorders.

### CROSS-REFERENCE TO RELATED APPLICATIONS

This application claims benefit of and priority to U.S. Ser. No. 60/692,048, filed on Jun. 16, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was funded, in part, by the National Institutes of Health. The Government of the United States of America may have certain rights in this invention.

### FIELD OF THE INVENTION

This invention pertains to the field of high throughput screening. A screening system comprising a reconstituted amyloid beta protein channel is disclosed.

### BACKGROUND OF THE INVENTION

Protein conformational diseases, including neurodegenerative (e.g., Alzheimer's, Huntington's, Parkinson's, prion encephalopathies, as well as familial British and Danish dementias (FBD, FDD)), systemic (e.g., type II diabetes, light chain amyloidosis) and other (e.g., cystic fibrosis) diseases result from protein misfolding that alters the three-dimensional (3D) conformation of the protein from native (often soluble) to non-native (often insoluble) folded structures (see, e.g., Temussi et al. (2003) Embo J., 22: 355-361; Dobson (2003) Nature, 426: 884-890; Selkoe (2003) Nature, 426: 900-904; Revesz et al. (2003) J. Neuropath. Exp. Neurol., 62: 885-898). Understanding such misfolding and the 3D conformations that induce pathophysiology and degeneration are one of the most important and yet challenging area of research (Temussi et al. (2003) Embo J., 22: 355-361).

One of the prevailing dogmas about these conformational diseases is that misfolded proteins assume fibrillar features termed amyloid that results in a gain-offunction and induces a pathophysiological cellular response by altering cell membrane composition and destabilizing cellular ionic homeostasis. Mechanisms underlying the formation of amyloid (amyloidosis) and its prevention have been studied extensively in the last few decades (see, e.g., Dobson (2003) Nature, 426: 884-890). Recent studies, however, indicate that fibrillar aggregates could simply be a storage mechanism and/or even be protective and that only globular (not fibrillar) conformations of amyloid proteins are sufficient to induce cellular degeneration and pathophysiology (Lin et al. (2001) FASEB J., 15: 2433-2444; Zhu et al. (2000) FASEB J., 14: 1244-1254; Bhatia et al. (2000) FASEB J., 14: 1233-1243; Walsh et al. (2002) Nature, 416: 535-539; Gibson et al. (2004) J. Neurochem., 88: 281-290; Bucciantini et al. (2002) Nature, 416: 507-511; Koistinaho et al. (2001) Proc. Natl. Acad. Sci., USA, 98: 14675-14680; Etcheberrigaray et al. (1994) Science, 264: 276-279).

Numerous studies have examined the mechanisms underlying globular peptide-induced cell dysfunction (see, e.g., Temussi et al. (2003) Embo J., 22: 355-361; Pollard et al. (1995) Cell Mol. Neurobiol., 15: 513-526; Kourie and Henry (2002) Clin. Exp. Pharm. Physiol., 29: 741-753; Kayed et al. (2004) J. Biol. Chem., 279: 46363-46366).

The deleterious effects of these globular proteins are proposed to be mediated either via their membrane poration as the key event followed by non-specific membrane leakage (Kayed et al. (2004) J. Biol. Chem., 279: 46363-46366; Green et al. (2004) J. Mol. Biol., 342: 877-887), or, most likely by specific ionic transport through ion channels (see, e.g., Kourie and Henry (2002) Clin. Exp. Pharm. Physiol., 29: 741-753; Lin et al. (1999) Biochemistry, 38: 11189-11196; Rhee et al. (1998) J. Biol. Chem., 273: 13379-13382; Kawahara et al. (2000) J. Biol. Chem., 275: 14077-14083; Arispe et al. (1993) Proc. Natl. Acad. Sci., USA, 90: 10573-10577; Hirakura et al. (2002) Amyloid 9: 13-2314) (for reviews see, e.g., Temussi et al. (2003) EMBO J., 22: 355-361; Pollard et al. (1995) Cell Mol. Neurobiol., 15: 513-526; Kourie and Shorthouse (2000) Am. J. Physiol., 278: C 1063-C 1087) that would destabilize ionic homeostasis. Indeed, amyloid peptides induce ionic conductances in both artificial membranes as well as in native cell plasma membrane (Lin et al. (2001) FASEB J., 15: 2433-2444; Etcheberrigaray et al. (1994) Science, 264: 276-279; Lin et al. (1999) Biochemistry, 38: 11189-11196; Rhee et al. (1998) J. Biol. Chem., 273: 13379-13382; Kawahara et al. (2000) J. Biol. Chem., 275: 14077-14083; Arispe et al. (1993) Proc. Natl. Acad. Sci., USA, 90: 10573-10577; Hirakura et al. (2002) Amyloid 9: 13-23). Very little is known, however, about the 3D structures of these globular peptides in the membrane. Lashuel et al. (2002) Nature, 418: 291-291, have recently shown “pore-like” annular structure for amyloidogenic protofibrils. However, these protofibrils were never associated with membranes (i.e., neither isolated from membrane complexes or reconstituted in membranes) and thus whether they form actual membrane pores was unknown.

Moreover, previous methods to investigate ion pore formation were limited. Traditionally, single channel ion currents are studied using the patch-clamp technique (Neher and Sakmann (1976) Nature, 260: 799-802; Sakmann and Neher (1983) Single Channel Recording, Plenum New York), in which a glass pipette filled with electrolyte is used to contact the membrane surface and measure ionic current. Recently, there has been a growing interest in chip-based patch clamping, using planar silicon microstructures. The aperture in a planar chip device has a lower background noise due to a lower series resistance and capacitance (Fertig et al. (2002) Appl. Phys. Letts., 81: 4865-4867) and the planar layout allows in situ measurements using AFM or fluorescence microscopy simultaneous with the electrical recording. However, these systems use whole cells that are too mobile to be useful for AFM imaging of ion channels at molecular resolution. Also, micrometer sized pores may be too large for high resolution imaging of reconstituted ion channels in bilayers. For such a study, a supported bilayer system with defined nanopores would be a more feasible option. Furthermore, traditional techniques are laborious and require precisely pulled pipettes that need to be positioned at the membrane interface using micromanipulators.

Several silicon wafer-based patch clamp systems have been designed and tested. Macroscopic ion channel activities in whole-cell systems have been studied using ion-milled glass substrates (Id.). Similarly, oocytes were patch-clamped to micromachined polydimethylsiloxane (PDMS) substrates (Klemic et al. (2002) Sigworth Biosensors & Bioelectronics, 17: 597-604). Silicon devices are perhaps the most versatile option to investigate single channel conductance, although other chip-based patch-clamp devices were proposed using silicon oxide coated nitride membranes (Fertig et al. (2000) Applied Physics Letters, 77: 1218-1220), polyimide films (Stett et al. (2003) Medical & Biological Engineering & Computing, 41: 233-240) and quartz substrates (Fertig et al. (2002) Biophysical J., 82: 161A-161A). To mimic the traditional patch pipettes, a silicon oxide micronozzle was developed (Lehnert et al. (2002) Appl. Phys. Letts., 81: 5063-5065) though no channel activity was observed due to a low electrical seal. Macroscopic channel activity has been observed using a silicon wafer based device (Pantoja et al. (2004) Biosensors & Bioelectronics, 20: 509-517). To improve the seal resistance, Teflon was deposited on silicon pores to produce a hydrophobic surface for bilayer attachment (Wilk et al. (2004) Appl. Phys. Letts., 85: 3307-3309). A multiple planar patch clamp system has been designed that uses lateral cell trapping junctions to reduce capacitive coupling and allows for multiplexed parallel patch sites (Seo et al. (2004) Appl. Phys. Letts., 84: 1973-1975).

These techniques, however, do not give information about the three-dimensional conformational states of ion channels related to their activity. Thus a need exists for techniques that can image the structural features of ion channels and recording their electrical activity simultaneously. For instance, transport of calcium ions through hemichannels has been demonstrated using AFM imaging on whole cell level (Quist et al. (2000) J. Cell. Biol., 148: 1063-1074), while the conformational differences between open and closed hemichannels were imaged using AFM after reconstitution of hemichannels in lipid bilayers (Thimm et al. (2005) J. Biol. Chem., 280: 10646-10654). Similarly, AFM has been successfully used to study the 3D structure of several types of amyloid ion channels related to protein misfolding disease (see, e.g., Example 2, herein, Quist et al. (2005) Proc. Natl. Acad. Sci., USA, 102: 10427-10432; Lin et al. (2001) FASEB J., 15: 2433-2444). However a direct correlation of the 3D structure and activity of single ion channels is yet to be demonstrated.

### SUMMARY OF THE INVENTION

The present invention relates to the discovery of a novel channel structure of human amyloid beta protein (AbP) in lipid membranes and a rapid, quantitative and specific assay for screening test compounds, such as drugs, ligands (natural or synthetic), proteins, peptides and small organic molecules for their ability to bind and block the membrane AbP channels. The invention further relates to screening and identifying therapeutically relevant compounds for treating Alzheimer's disease and other disorders.

Thus, in certain embodiments, this invention provides a device for screening for molecules that alter ion channel activity. The device typically comprises a lipid bilayer attached to a solid support, where the lipid bilayer contains one or more ion channel proteins. In certain embodiments the solid support comprises one or more nanopores (e.g., 1, 2, 4, 8, 10, 20, 50, 100, 500, 1000, or more nanopores). In certain embodiments embodiments, the nanopores range in size from about 10 nm to about 400 or 500 nm in diameter, preferably from about 20 nm to about 200 nm in diameter, more preferably from about 50 nm to about 100 nm in diameter. In certain embodiments the nanopores range in diameter from about 5 nm, 10 nm, 20 nm, 30 nm, or 50 nm to about 500 nm, 400 nm, 300 nm, 250 nm, 200 nm, 150 nm, 100 nm, 90 nm, 80 nm, or 70 nm. In various embodiments the nanopores penetrate through a surface having a thickness of about 400 nm or less, preferably about 300 nm or less, and more preferably about 200 nm or less. In certain embodiments the nanopores are formed in a membrane and/or a silicon wafer and are optionally disposed so that one or more nanopores aligns with an ion channel. The device can further comprise a fluid reservoir on one side and/or on the other side of the lipid bilayer. In certain embodiments the one or more ion channel proteins are selected from a group consisting of a calcium channel, a sodium channel, a potassium channel, a chloride channel, and a magnesium channel. In certain embodiments the one or more ion channel proteins comprise amyloid proteins (e.g., AbP channel proteins). The device can optionally further comprise a means for detecting alteration of channel conformation in response to contact with a compound. Such means include, but are not limited to an atomic force microscope (AFM) probe or a scanning probe microscopy (SPM) probe. In various embodiments the device can comprise means to provide a measure of ion channel conductivity. In various embodiments the means provides both a measure of channel conductivity and channel protein conformation. In various embodiments the means provides a measure of channel conductivity and additionally comprises an AFM or an SPM. In various embodiments the device comprises a plurality of different channels (e.g. at least 2, preferably at least 5, more preferably at least 10 or 20, and most preferably at least 50 or 100 different channels). In certain embodiments a plurality of the channels are each aligned with a pore in the solid support.

Also provided is a method of screening a test agent for the ability to alter conductivity or conformation of an ion channel (e.g. an AbP channel). The method typically involves contacting a device as described above with a test agent; and detecting a change in conformation and/or conductivity of a channel in response to the contact with the test agent. In certain embodiments change in conformation is measured using AFM or SPM. In various embodiments the change in conductivity is measured using an AFM or SPM tip as an electrode. In certain embodiments the change in conformation and change in conductivity are measured simultaneously.

This invention also provides a method of screening test agents for the ability to alter pore conformation or conductance by amyloid proteins. The method typically involves providing a lipid bilayer comprising a pore comprising one or more amyloid proteins; contacting the lipid bilayer with a test agent; and detecting a change in the conformation and/or conductance of the pore, where a change in conformation and/or conductance indicates that the test agent alters pore conformation or conductance.

In certain embodiments this invention provides an AFM or SPM having an integrated carbon nanotube cantilever and tip. This invention also provides a carbon nanocone, or an AFM or SPM having a carbon nanocone tip. The nanocone typically comprises a high-aspect ratio carbon nanotube structure substantially lacking a catalyst at the tip. In certain embodiments the nanocone has a cone angle of less than about 15 degrees, preferably less than about 10 degrees, more preferably less than about 5 degrees. In certain embodiments the nanocone has an aspect ratio (height:base) of at least about 5:1, preferably at least about 10:1, more preferably at least about 12:1. In certain embodiments the nanocone has a tip radius of less than about 10 nm, preferably less than about 5 nm, more preferably less than about 3 nm.

Also provided is a method of fabricating a nanocone. The method typically comprises a resist-free e-beam induced deposition (EBID) of carbon masks combined with electric-field-controlled CVD growth. In certain embodiments the method utilizes EBID carbon patterns as dry etching masks.

Definitions.

Ion channels are proteins in cell membranes that act as pores to permit the passage of charged species (ions) across the cell membrane (e.g., a lipid bilayer). In certain embodiments the ion channels have the ability to open or close in response to specific stimuli and thus allow for gating of ions in and out of enclosed subcellular compartments and/or whole cells. Ion channel proteins can be referred to by the type of ion they pass. Thus, for example, a calcium channel, is an ion channel that selectively or preferentially allows the passage of calcium ion (Ca²⁺) through a membrane.

An ion channel protein is a protein that is a component of an ion channel.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The terms “AbP” or “AβP” refer to human amyloid beta protein.

### BRIEF DESCRIPTION OF THE DRAWINGS

FIG. 1 shows CD spectra of mutant amyloid molecules in solution. CD spectrometry analysis of ABri, ADan, α-synuclein, amylin, SAA, and Aβ(1-40) in 5 mM Tris (pH 7.4) was carried out on a Jasco J-720 spectropolarimeter at 1-nm intervals over the wavelength range 190-260 nm at 24° C. in a 0.1-cm path-length cell. Results are expressed in molar ellipticity (deg cm² mol⁻¹.).

FIG. 2 shows Electrophoresis of ABri, ADan, α-synuclein, amylin, SAA, and Aβ(1-40) on 16.5% SDS-PAGE under reducing conditions. The right lane shows peptide in aqueous solution; the left lane shows peptide in DOPC membrane after solubilization in 2% SDS. Positions of molecular mass markers are indicated on the left. Amylin and Aβ(1-40) were cross-linked both in solution and in membrane. In solution, for all peptides the monomers are observed. Small amounts of dimers are observed in solution for Aβ(1-40), ABri, and amylin. In the membrane, besides monomers and dimers, trimers are observed for amylin and Aβ(1-40), and tetramers are observed for ABri, amylin, Aβ(1-40), and α-synuclein. Also observed are pentamers for amylin and Aβ(1-40); hexamers for α-synuclein, SAA, ADan, amylin, and Aβ(1-40); and heptamers and octamers for α-synuclein and SAA.

FIG. 3 shows single-channel records of amyloid channels. Current traces as a function of time under voltage-clamp conditions are shown. Current jumps corresponding to the opening or closing of individual ion channels can be observed for all six amyloid peptides. Solutions contained 100 mM KCL (except B, which contained 10 mM KCl, and F, which contained 1 M KCl), buffered to pH 7.4. Peptide concentrations were as follows. (A) Aβ(1-40): 21 μg/ml, V=−30 mV. (B) Amylin: 3 μg/ml, V=−50 mV. (C) ABri: 50 μg/ml, V=−50 mV. (D) ADan: 100 μg/ml, V=−50 mV. (E) NAC: 15 nM, V=−50 mV. (F) SAA: 1 μg/ml, V=−60 mV.

FIG. 4 shows AFM images of freshly dissolved peptide molecules. Green arrows indicate surface-adsorbed peptides with a width of 8-12 nm and a height of 1-2 nm (consistent with the size of monomers). Arrows indicate higher-order oligomers and clusters. For ABri, most observed features are monomers and dimers; for other peptides, larger multimers and aggregates are observed. For ADan and amylin, the amount of monomers adsorbed is small, and mostly multimers and clusters are observed. In one experiment for α-synuclein, fiber-like structures were observed, indicated by dotted lines. Peptides were adsorbed on mica for 20-40 min and then washed to remove unadsorbed peptides and imaged in buffer (see Materials and Methods in Example 2). (Scale bars: 500 nm for ADan and 100 nm for all other peptides.)

FIG. 5 shows AFM images of amyloid peptides reconstituted in membrane bilayers. _{Inset} shows lipid bilayer with thickness of ˜5 nm. For Aβ(1-40), ADan, and α-synuclein, channel-like structures with a central pore can be easily resolved. For ABri, SAA, and amylin, the central pore is only resolved on some multimer structures. Arrows indicate locations where annular structures can be observed clearly. For those indicated channels, channel sizes (outer diameter) are 16 nm for Aβ(1-40), 14 nm for ABri, 14 nm for ADan, 16 nm for α-synuclein, 12 nm for SAA, and 15 nm for amylin. (Scale bars: 100 nm.)

FIG. 6 shows individual channel-like structures at high resolution. Two examples are shown for each molecule, in which a central pore can be observed. The number of subunits observed protruding from the surface varies from four to eight subunits. Resolution of AFM images is not enough to resolve individual subunit structures. [Image sizes are 25 nm for Aβ(1-40), 25 nm for α-synuclein, 35 nm for ABri, 20 nm for ADan, 25 nm for amylin, and 20 nm for SAA.]

FIG. 7 schematically illustrates the cross-section of a conventional probe.

FIG. 8, panels A, B, and C illustrate processing steps for a probe as described herein.

FIG. 9 illustrates a schematic of a probe assembly with a detection mechanism.

FIG. 10 illustrates a vertically grown nanotube.

FIG. 11 illustrates a vertically grown nanotube with an added CNT segment grown at an angle.

FIG. 12, panels A-D show carbon nanotube tips of different shapes.

FIG. 13, panels A, B, and C illustrate a mechanism of growing carbon nanotubes at an angle.

FIG. 14 illustrates examples of carbon nanotube growth manipulation.

FIG. 15 Panel A: Tapping mode AFM image of arrays of nanopores (left). The pores are produced in the silicon nitride windows using electron beam lithography and range in size from 50 to 100 nm. Panel B: After deposition of lipid vesicles, a bilayer is formed covering the pores (center, imaged under buffer). The bilayer has several holes; the cross section on a hole in the bilayer shows a depth of 5.5 nm, the typical thickness of a lipid bilayer. Scale bars 1 micron. Panel C shows a schematic of the liquid cell AFM setup imaging the nitride window.

FIG. 16 shows force distance curves acquired over an alignment mark that is partially covered by a lipid bilayer. Crosses indicate position of force curves acquired on the substrate (top), edge of the mark (middle) and a bilayer suspended over the mark (bottom). The increased z-distance from point of contact to pre-set deflection indicates a softer sample (arrows). The inset shows a stable suspended bilayer imaged repetitively using 1.5 nN force. Scale bar 500 nm.

FIG. 17A shows combined AFM height and current imaging. The left images show topography of the same FIB milled pore imaged repeatedly with varying bias potential, −2.0 V (top), −1.0 V (middle), and −0.5 V (bottom). The right images are current images, a distinct current signal is observed over the pore. The amount of current is shown in the cross sections of the current images, 214 pA at −2.0 V, 65 pA at −1.0 V, and 5 pA at −0.5 V. FIG. 17B shows a schematic setup with the platinum electrode in buffer under the nitride window. Scale bar 1 micron.

FIG. 18A shows IV curves collected 10 seconds (top), 2 minutes (middle) and 4 minutes (bottom) after addition of Gramicidin to the lipid bilayer sample suspended over the nanopore. IV voltage range −0.5 to 0.5 Volt. Conductance increases from 0.025 nS (bilayer only) to 0.25 nS, 0.5 nS and 1 nS respectively. FIG. 18B shows a schematic setup, and FIG. 18C shows a schematic of the FIB induced nanopore with deposited oxide and bilayer.

FIG. 19, panels A-F, schematically illustrates a fabrication procedure. Panel A: Device layer (gray), buried oxide layer (cross-hatched) and handle wafer (gray). B) thermal oxide (cross-hatched) and silicon nitride (white) added as masking layer. C) Photolithography leaves etched pyramidal shaped pit. D) Removal of masking layers and nano pore fabrication using FIB followed by oxide deposition. For details see Example 3. E) Backside of wafer after etching shows 200×200 micron pyramidal opening. F) Nanopore created by FIB, diameter 70 nm.

FIG. 20 schematically illustrates a setup for AFM and combined electrical recording. Images of topography and current are acquired simultaneously.

FIG. 21, panels A-D, show a schematic of the resist-free fabrication technique for a single CNC based AFM tip. Panel A: Catalyst deposition by e-beam evaporation. Panel B: E-beam induced deposition of a carbon dot mask. Panel C: Metal wet etching and the removal of a carbon dot. Panel D: CVD growth of a CNC probe.

FIG. 22, panels A-D, show schematics and SEM of carbon nanotubes and nanocone formation. Panel A: Schematic of an equidiameter carbon nanotube growth. Panel B: SEM image of equidiameter CNTs grown on an Si substrate at a dc bias of 450 V for 20 min. Panel C: Schematic of the gradual reduction of a catalyst particle at the CNT tip by sputtering and accompanying reduction in the CNT diameter. Panel D: SEM image of the CNC with the catalyst particle completely removed at 550 V for 20 min.

FIG. 23, panels A and B, show an SEM image of a single CNC probe. Panel A: Top view SEM image of the single CNC probe grown near the edge of a cantilever (low-magnification, 30° tilted view). Panel B: Side view SEM image of the CNC probe. Inset: TEM image of the CNC tip.

FIG. 24, panels A-F shows a comparison of AFM imaging using a nanocone and a conventional tip. Panel A: Copper film AFM image. Panel B: Zoom-in Cu film AFM image. Panel C: AFM image of a PMMA line pattern by a conventional Si pyramid tip. Panel D: Image of the same PMMA pattern by a CNC tip. Panel E: The height profile of image panel C. Panel F: The height profile of image panel D.

### DETAILED DESCRIPTION

This invention pertains to the discovery that certain protein misfoldings, in particular the misfolding of various channel proteins (e.g., ion channel proteins) is implicated in the etiology of various pathologies. Protein conformational diseases, including, but not limited to Alzheimer's disease, Huntington's disease, Parkinson's disease, and the like, result from protein misfolding, giving a distinct fibrillar feature termed amyloid. Recent studies have shown that only the globular (not fibrillar) conformation of amyloid proteins is sufficient to induce cellular pathophysiology. However, the 3D structural conformations of these globular structures, a key missing link in designing effective prevention and treatment, has remained undefined.

By using atomic force microscopy, circular dichroism, gel electrophoresis, and electrophysiological recordings, we showed that an array of amyloid molecules, including amyloid-β(1-40), α-synuclein, ABri, ADan, serum amyloid A, and amylin undergo supramolecular conformational changes. In reconstituted membranes, they form morphologically compatible ion-channel-like structures and elicit single ion-channel currents. These ion channels would destabilize cellular ionic homeostasis and hence induce cell pathophysiology and degeneration in amyloid diseases.

Thus ion channels and ion channel proteins particularly those comprising amyloid proteins provide targets to screen for agents that modulate (e.g., inhibit, or stabilize/upregulate) pore formation and such agents are expected to provide effective lead compounds for the development of therapeutics for the treatment of protein conformation pathologies.

In certain embodiments it was discovered that the fibril formation of amyloid beta protein (AbP) is not required for AbP-induced cellular toxicity and the non-fibrillar form of AbP, at physiological levels, can induced cell degeneration. Moreover this damage is not via the mechanisms of oxidative damage or binding of tachykinin receptors as previously proposed.

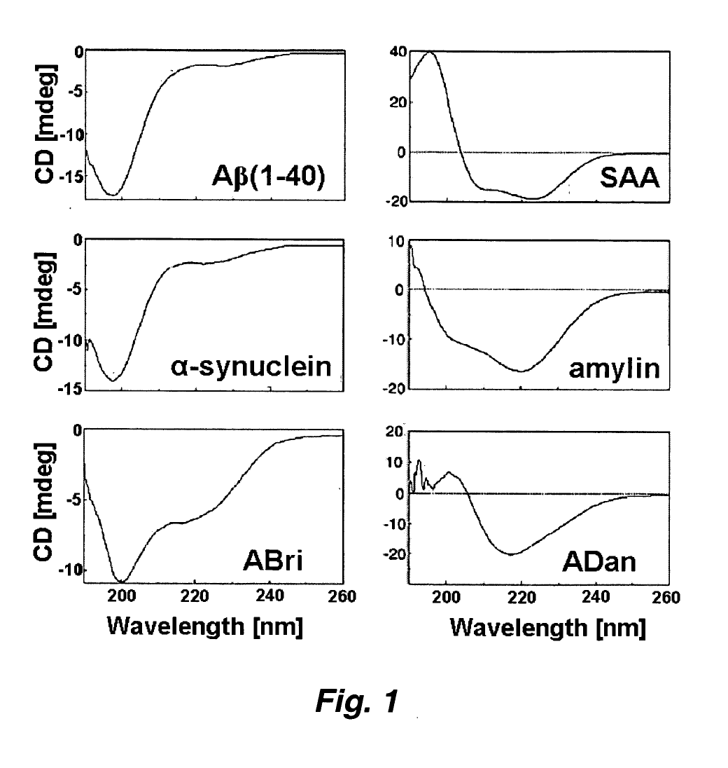

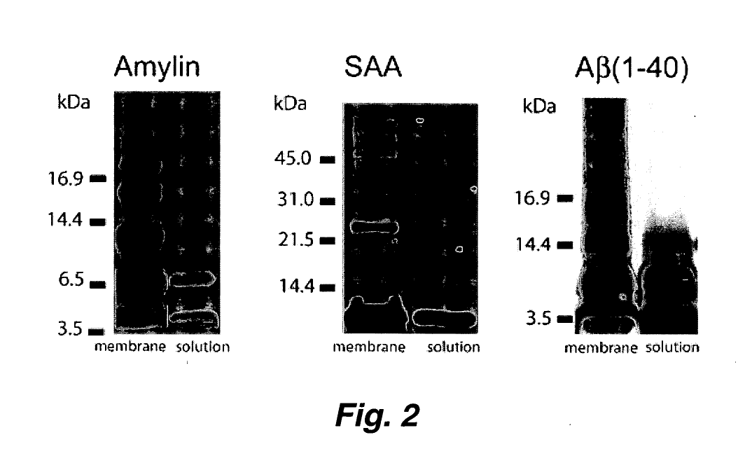

We have shown that the 43-residue AbP form channel-like structure in lipid membranes. We have seen two specific three dimensional forms of the channel, hexameric and tetrameric channels (FIG. 1), which is also consistent with the results of various biochemical assays (FIG. 2).

In cultured cells, blocking Ca²⁺ influx through AbP ion channels, by the addition of Zn²⁺ or removal of extracellular Ca²⁺, inhibited AbP-induced toxicity. Consistent with these discoveries, we have shown that AbP ion channels mediate Ca²⁺ influx when incorporated into unilaminar liposomes, and the Ca²⁺ influx was blocked by Zn²⁺ and an anti-AbP antibody.

In various embodiments the present invention relates to rapid, quantitative and specific assays for screening test compounds, such as drugs, ligands (natural or synthetic), proteins, peptides and small organic molecules for their ability to bind and block, or alternatively, in certain cases to stabilize, the membrane ion channels comprising one or more amyloid proteins (e.g., AbP channels). In certain embodiments modulation of AbP channels will prevent cellular calcium imbalance and thereby prevent or mitigate symptoms of Alzheimer's disease

The present invention also relates to the drugs, ligands, proteins, peptides and small organic molecules identified by the screening assay of the present invention as capable of inhibiting membrane AbP channels.

The invention is based, in part, on our discovery and demonstration that AbP form channel-like structure in lipid membranes. We have seen two specific three dimensional forms of the channel, hexameric and tetrameric channels (FIG. 1), which is also consistent with biochemistry assay (FIG. 2)

Design of the Assay.

In various embodiments this invention contemplates assays and devices that use liposomes or planar lipid bilayers with incorporated ion channel proteins (e.g., AbP channels) as target to screen for therapeutically relevant molecules for treating Alzheimer's disease and other disorders. The AbP channel proteins, include, but are not limited to all peptide channels from by proteolytic product of β-amyloid precursor protein (AbPP), such as AbP₁₋₂₅, AbP₁₋₃₉, AbP₁₋₄₀, AbP₁₋₄₂, AbP₁₋₄₃, and the like.

In certain embodiments one or more test agents are screened for their ability to bind, preferably to specifically bind, one or more ion channel proteins, preferably amyloid ion channel proteins when present in a lipid bilayer. In various embodiments the amyloid ion channel proteins can be introduced into an isolated bilayer (e.g. a bilayer attached to a solid support), into a liposome comprising a bilayer, into an oocytes comprising a bilayer (e.g., a Xenopus oocytes), into a cell, and the like.

Binding of the test agent(s) to the amyloid channel protein(s) can be detected by any of a number of methods known to those of skill in the art. For example, in certain embodiments, the test agent(s) are labeled with a detectable label (e.g., a fluorescent label, a calorimetric label, a radioactive label, a spin (spin resonance) label, a radiopaque label, etc.). The membrane comprising the amyloid channel protein(s) is contacted with the test agent(s), typically washed, and then the membrane is screened for the detectable label indicating association of the test agent with the amyloid channel protein. In certain embodiments a secondary binding moiety (e.g. bearing a label) is used to bind and thereby label the bound test agents, or to bind the amyloid channel proteins in which case association of the label on the secondary agent with the label on the test agent indicates binding of the test agent to the amyloid channel protein. In the latter case, in certain embodiments, the label on the test agent and the label on the secondary agent can be labels selected that undergo fluorescent resonance energy transfer (FRET) so that excitation of one label results in emission from the second label thereby providing an efficient means of detecting association of the labels.

In certain embodiments, the assay is a competitive assay format. In such assays, a “competitive” agent (e.g., antibody, small organic molecule, etc.) known to bind to the amyloid channel protein is also utilized. The competitive agent can be labeled and the amount of such agent displaced when the bilayer containing the amyloid protein(s) is contacted with a test agent provides a measure of the biding of the test agent. Methods of detecting specific binding are well known and commonly used, e.g. in various immunoassays. Any of a number of well recognized immunological binding assays (see, e.g., U.S. Pat. Nos. 4,366,241; 4,376,110; 4,517,288; and 4,837,168) are well suited to detection of test agent binding to amyloid channel proteins in a lipid bilayer. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

In certain embodiments binding of test agents can be detected by detecting alterations (e.g., decrease) of ion (e.g., Ca²⁺) uptake by a cell, oocytes, or liposome in the presence of the test agent(s)-uptake in to the liposomes. This can readily be detected using for example a radio-isotope (e.g., 45Ca²⁺) or a calcium sensitive dye (e.g., arsenazo III (AIII), Fura-2, Fluo-3, Fluo-4, Calcium green, etc.).

In certain embodiments the modulation of amyloid channels by test agent(s) can be detected by monitoring changes in ionic channel conductances in cells or oocytes, or in liposomes, lipid layers, or other ex vivo systems. Methods of detecting ion channel conductivity are well known to those of skill in the art. Traditionally, single channel ion currents are studied using the patch-clamp technique (see, e.g., Neher and Sakmann (1976) Nature, 260: 799-802; Sakmann and Neher (1983) Single Channel Recording, Plenum New York), in which a glass pipette filled with electrolyte is used to contact the membrane surface and measure ionic current. Various chip-based patch clamping methods are also known (see, e.g., Fertig et al. (2002) Appl. Phys. Letts., 81: 4865-4867).

In certain embodiments this invention contemplate the use of an ex vivo system comprising two chambers separated by a lipid bilayer, that contains an amyloid protein ion channel. The conductance across the lipid bilayer is monitor continuously. This device can be used to assay molecules that block or modulate the activity of, for example, AbP channels. Various features of such a device are described in more detail below.

In certain embodiments alterations of channel conductivity and/or conformation can be measured using scanning probe microscopy (SPM) and/or atomic force microscopy (AFM). Methods of detecting protein conformation changes using SPM or AFM are known to those of skill in the art (see, e.g., Miller and Engel (2001) RIKEN Rev., 36: 29-31).

Chip-Based SPM/AFM Devices for Screening Channel Proteins Conformation Changes.

In certain embodiments, this invention contemplates chip-based supported bilayer systems for screening test agents for their ability alter ion channel protein conformation and/or conductance. In various embodiments the device comprises a lipid bilayer attached to (disposed on) a support. The support is typically a microfabricated support (e.g., micromachined using photolithographic methods and/or various ion beam etching methods).

In certain embodiments the support comprises one or more nanopores. The nanopores typically range in size from about 10 nm to about 400 nm, preferably from about 20 nm to about 200 nm in diameter, more preferably from about 50 to about 100 nm in diameter. In certain embodiments the nanopores range from about 10 nm, 20 nm, 30 nm, 50 nm, or 70 nm in diameter, to about 400 nm, 300 nm, 250 nm, 200 nm, 150 nm, or 100 nm in diameter. Support can be a rigid support, or in certain embodiments, a membrane support.

The nanopores can be produced in electron beam lithography as well as by using a finely focused ion beam. Thermal oxide can used to shrink pore sizes, if necessary and to create an insulating surface.

The chips with well defined pores can be mounted on a double chamber plastic cell recording system allowing for controlling the buffer conditions both above and below the window (membrane). In addition the system can be oriented to permit use of an AFM or SPM tip to measure ion channel protein conformation. The AFM or SPM tip can also function as an electrode and with a second electrode (e.g., a platinum wire) under the membrane window conductance across lipid bilayers that are suspended over the pores can readily be measured. The probes can, optionally, further comprise a nanotubes that functions as an electrode. The fabrication and use of such a system is illustrated herein in Example 3. Using the teaching provided herein, other variants of such systems will readily be available to one of skill in the art.

Microfabricated spm Probes with Integrated Carbon Nanotube Cantilever and Tip

As indicated above, in certain embodiments, this invention utilizes microfabricated SPM or AFM probes preferably having an integrated carbon nanotube cantilever and tip.

With the growing field of scanning probe microscopes (SPMs), demand for new probes with special cantilever and tips is also increasing to meet the requirements of various applications. Most of these probes are made of either silicon or silicon nitride with similar material of the tip. For some applications the tip is coated with different materials to perform measurements such as electrical, magnetic, etc. Obtaining a very high resolution images has always been a goal for the scientific community. Since silicon is very brittle, the sharp tips do not last very long on hard surfaces. On the other hand the soft samples such as living cells have risk of getting damaged by the hard tip. Repeatability of the data with the same tip is desirable to make comparative and reliable studies. Attachment of nanotube tips on existing silicon tips has offered partial solution to these problems. At times, however, the cantilevers made of silicon and silicon nitride break too easily and as a consequence validation important research data becomes impossible. The material and shape of such cantilever with a very sharp tip remains an area of further development.

In certain embodiments this invention provides methods of manufacturing SPM/AFM probes that can, optionally, have, both, cantilever and tips made out of carbon nanotubes or similar materials. The process of manufacturing, presented in this invention, as shown in the attached figures, utilizes nano-fabrication technology in conjunction with the carbon nanotube growth process. The following features of these probes make them unique in their performance: They have a very low spring constant; they have a low squeeze film damping effect in air; they are ideal for imaging in liquid, they have a sharp tip, and a long lifetime.

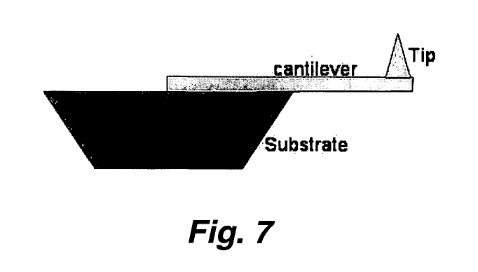

FIG. 7 shows cross-sectional view of a conventional SPM probe with integrated cantilever and tip. The probe has three parts: a substrate, a cantilever and a tip. The performance and the applications of a given probe are determined by the cantilever and the tip. The substrate facilitates handling and mounting of the probe assembly in the scanning probe microscopes.

In certain embodiments the methods of this invention involve growing a thick and vertical carbon nanotube (CNT) out of the silicon surface and using it as a cantilever. The tip made out of CNT also, can be attached at the end of the cantilever. A CNT reflector can then be grown near the end of CNT cantilever to facilitate imaging the surface through laser detection. In various embodiments the fabrication of silicon substrate and CNT cantilever growth is a batch fabrication process. The following sections briefly describe the process and variations of fabricating the device.

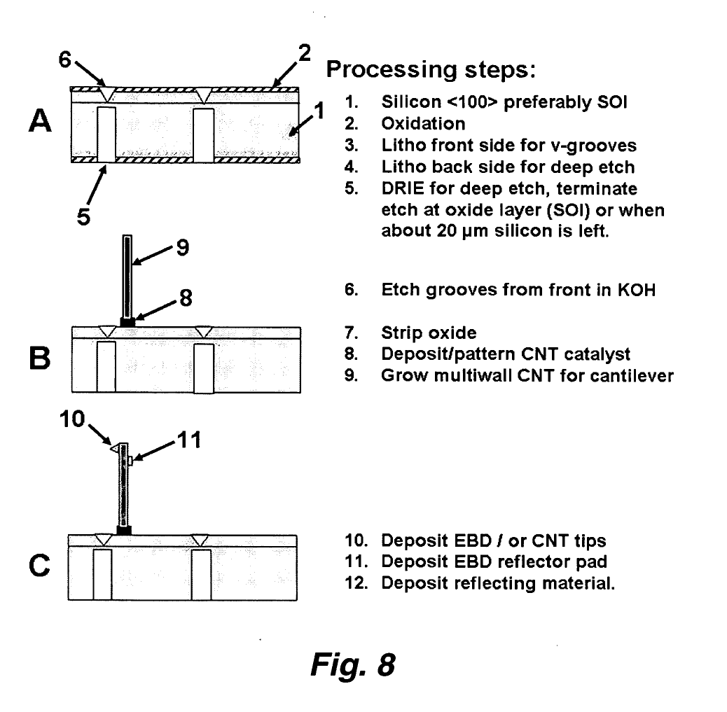

FIG. 8, panel A, shows the cross-sectional view of the silicon substrate formed after series of standard microfabrication processing steps mentioned in the diagram. The multiple substrates formed in the process can be separated after the carbon nanotube 9 growth as shown in FIG. 8, panel B. The individual substrates get separated at the deep groove 5 and v-groove 6. A gentle touch is sufficient to separate the substrates as they are held by about 20 um thick silicon membrane.

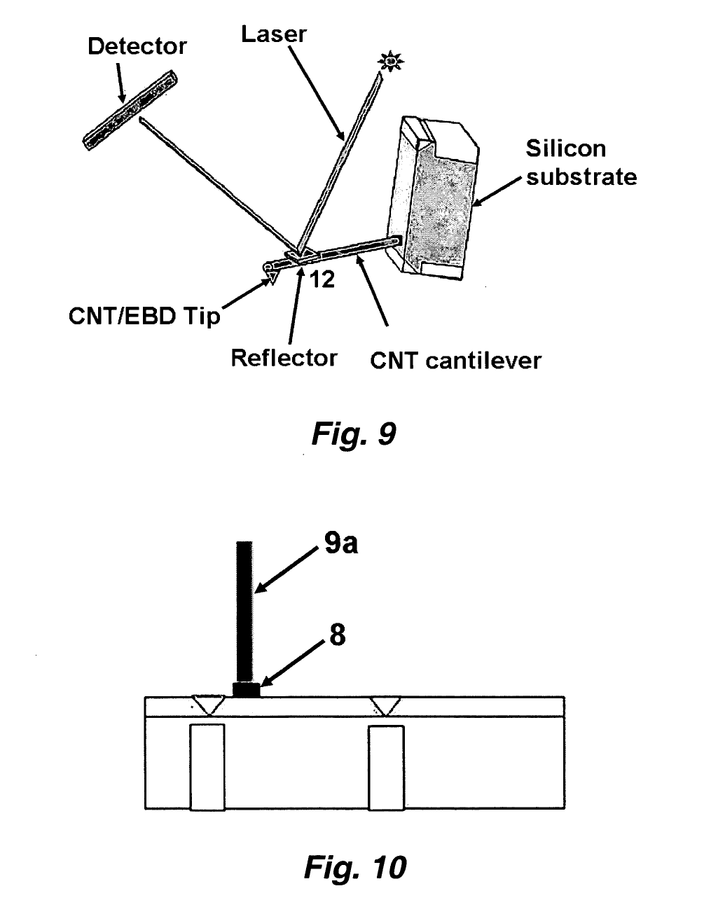

FIG. 8, panel C, shows a cross-sectional view of the completed device with CNT tip and CNT reflector. The reflector is coated with a metal layer to make the laser bounce from the source to the detector. A schematic of the probe assembly with laser detection system is shown in FIG. 9.

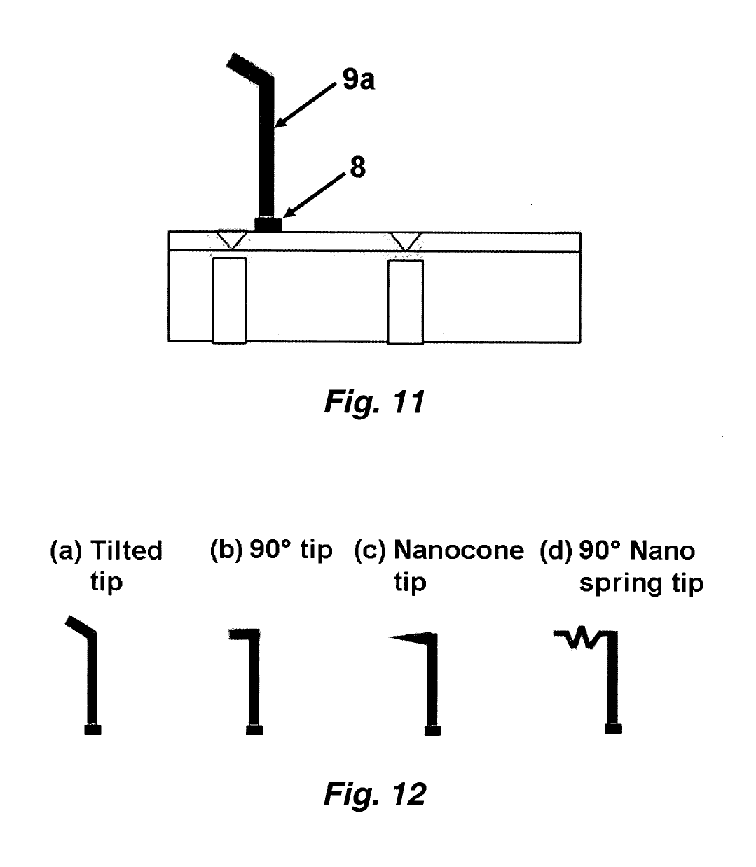

The CNT growth can be manipulated to make varieties of tips. FIG. 10 shows a vertical CNT 9a grown on silicon substrate having a catalyst 8. A segment of CNT as a tip is added at an angle at the free end of the CNT cantilever (FIG. 11). The added segments can be made in different shapes and dimensions for different applications as shown in FIG. 12, panels A-D.

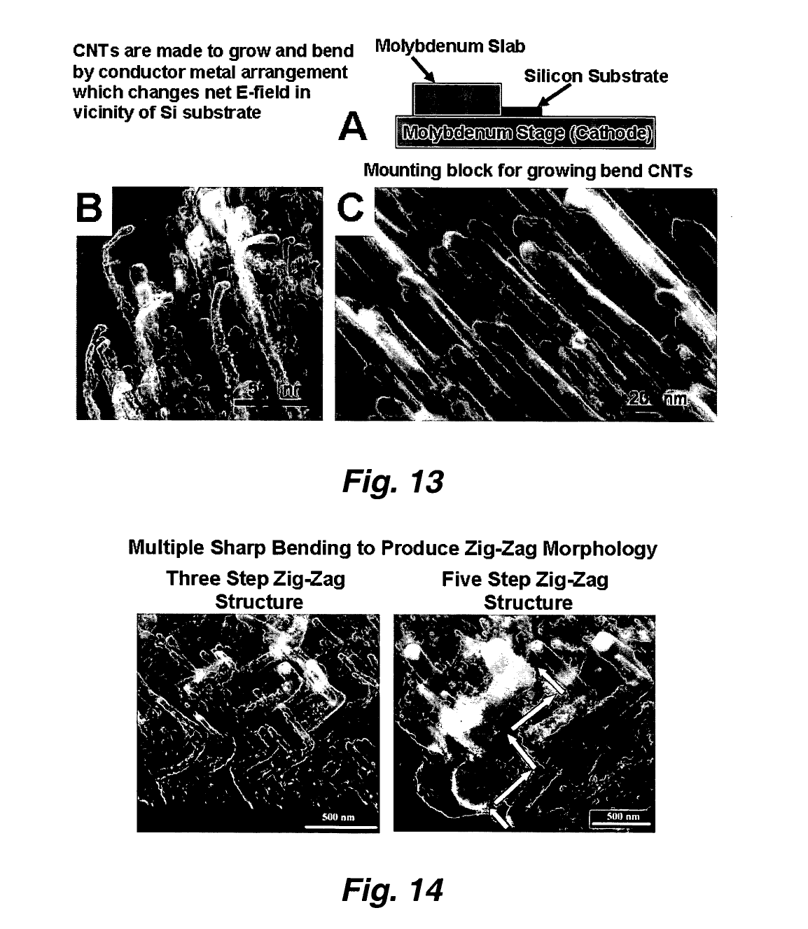

The precise bending of the CNT can be achieved by controlling the direction of the electric field during the growth of CNT. FIG. 13, panel A, shows a mounting block for the silicon substrate with catalyst to grow CNT at an angle. FIG. 13, panels B and C show CNTs grown at an angle in such system. The CNT growth angle can be controlled and manipulated with sharp bends to make zig-zag structures leading to spring tips as shown in FIG. 14.

The apex of the CNT tip can be flat or sharp (e.g., 1-2 nm).

The tips described herein, have a very low spring constant and can be used, for example in the fabrication of microcantilever arrays for biosensors and the like. In addition, the CNT cantilevers with sharp tips can be used for deterging changes in pore conductivity or conformation and can also be used for high resolution images in life science and materials studies. The tilted tips are also useful for sidewall roughness measurement. The flat apex of the CNT tip will provide reproducibility and long life time.

V. High Throughput Screening for Agents that Modulate Ion Channel Protein Conformation and/or Conductivity.

In various embodiments the above assays can be implemented in a parallel array for simultaneous screening of multiple different molecules. Thus, for example, small unilaminar liposomes can be cross-linked to attach onto a solid support, and implanted in a multi-array system, such as a fabricated silicon chip or a multi-well system. Similarly, planar lipid bilayer with incorporated ion channels can be absorbed on a solid support, such as multi-well plates or fabricated chips. Under such implementation, multiple target compounds can be simultaneously tested, e.g. in a high throughput screening (HTS) format.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used directly in the desired application.

A) Combinatorial Chemical Libraries for Modulators Ion Channel Conformation and/or Conductivity.

The likelihood of an assay identifying an agent that modulates ion channel conformation and/or conductivity is increased when the number and types of test agents used in the screening system is increased. Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries for Modulators of Ion Channel Conformation and/or Conductivity.

Any of the assays for agents that modulate ion channel conformation and/or conductivity described herein are amenable to high throughput screening. Binding assays, for example, are well known and U.S. Pat. No. 5,559,410 discloses high throughput screening methods for protein binding, while U.S. Pat. Nos. 5,576,220 and 5,541,061 disclose high throughput methods of screening for ligand/antibody binding.

Moreover the chip-based devices described herein are well suited to high-throughput screening. Robotics systems for manipulating reagents and the like in conjunction with such assays are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

Other Ion Channel Proteins.

While the devices described herein are illustrated with respect to amyloid proteins that form ion channels, it will be appreciated that the devices are suitable for use, e.g. for screening test agents for the ability to increase, decrease, or block channel conductance of essentially any ion channel. Ion channels include, but are not limited to a calcium channel, a sodium channel, a potassium channel, a chloride channel, a magnesium channel, and the like. Protein constituents of various calcium, sodium, potassium, chloride, magnesium channels are known to those of skill. In addition, pathological states attributed to the dysfunction of these channels and particular proteins comprising such channels are also known to those of skill in the art.

For example, various illustrative chloride channels include, but are not limited to voltage gated chloride channels (CLC), including, but not limited to CLC-1, CLC-2, CLC-3, CLC-4, CLC-5, CLC-6, CLC-7, CLC-O, ClC-K/barttin channels (e.g., CLCN-KA, CLCN-KB), chloride intracellular channels CLIC-1, CLIC-2 CLIC-3 CLIC-4 CLIC-5, etc., calcium activated chloride channels (CLAC1, CLAC2, CLAC3, etc.), and the like. Pathologies associated with dysfunctional chloride channels include but are not limited to myotonia congenita (CLC-1), Myotonic Dystrophy (DM1; DM2), Epilepsy (CLC-2), Renal tubular disorders (CLC-5), Bartter's syndrome (CLC-KB), cystic fibrosis (epithelial chloride channel), osteopetrosis, etc.

Various illustrative sodium channels include, but are not limited to voltage-gated Na⁺ channels, (e.g., SCN1, SCN1ASCN2A1, SCN2A2, SCN3A, SCN4A, SCN5A, SCN7A, SCN8A (PN4), SCN9A (PN1), SCN10A, SCN11A, SCN1B (β1), SCN2B (β2), SCN3B, SCN4B), non-voltage-gated Na+ channels (e.g., epithelial sodium channel, degennerins, etc.), sodium/hydrogen exchanges (e.g., NAH1, NAH2, NAH3, NAH4, NAH5, SLC9A6, SLC9A7, etc.), SLC5A, SLC24, and the like. Pathologies associated with dysfunctional sodium channels include but are not limited to, hyperkalemic periodic paralysis, paramyotonia, myotonia, myasthenia, long qt syndrome 3, progressive cardiac conduction defect (PCCD2; Lenegre-Lev disease), congenital non-progressive heart block, idiopathic ventricular fibrillation, congenital sick sinus syndrome (SCN5A), hyperkalemic periodic paralysis, hypokalemic periodic paralysis, paramyotonia congenita, myotonia fluctuans, myotonia permanens, acetzolamide-responsive myotonia, malignant hyperthermia, myasthenic syndrome, multifocal motor neuropathy, acute motor axonal neuropathy etc.

Various illustrative calcium channels include, but are not limited to, voltage-gated Ca⁺⁺ channels (e.g., N-type, P-type, L-type, Q-type, R-type, P-type, etc.), ligand-gated Ca⁺⁺ channels (e.g., Ca⁺⁺ transporting ATPase), capacitive Ca++ entry channels, Intracellular activation channels, calcium sensors, and the like. Pathologies associated with dysfunctional sodium channels include but are not limited to, hypokalemic periodic paralysis (CACNL1A3 α1S subunit), malignant hyperthermia (CACNL1A3 α1S subunit), long QT syndrome with syndactyly (Timothy syndrome), X-linked congenital stationary night blindness, familial hemiplegic migraine, juvenile myoclonic epilepsy, granulomatous myopathy, brody myopathy, Darier-White disease: Keratosis follicularis, etc.

Various illustrative potassium channels include, but are not limited to, voltage gated potassium channels, inwardly rectifying potassium channels (e.g. (Kir channels, KCNK family, KCNJ family, KCNH family, KCNM family, etc.), delayed rectifier K⁺ channels, Ca⁺⁺ sensitive K⁺ channels (e.g. BK, IK, SK), TP-sensitive K⁺ channels, Na⁺ activated K⁺ channels, and the like. Pathologies associated with dysfunctional potassium channels include but are not limited to, atrial fibrillation, short QT syndrome. episodic ataxia/myokymia syndrome, myokymia & benign neonatal epilepsy, etc.

The foregoing ion channels, associated proteins, and pathologies are intended to be illustrative and not limiting. Other ion channels and ion channel proteins will be known to those of skill in the art.

Kits

In certain embodiments, this invention provides kits for practicing the various methods described herein. The kits can include, for example, the assay devices described herein. In various embodiments the assay device is a chip based device and is, optionally, provided in a format compatible with a commercially provided reader.

Where the microcantilever device incorporates reservoirs, the reservoirs can, optionally, contain one or more buffers, labels, and/or bioactive agents as required. In certain embodiments the bioactive agent or other agent is provided in a dry rather than a fluid form so as to increase shelf life.

The kits can optionally further comprise buffers, syringes, sample collectors and/or other reagents and/or devices to perform one or more of the assays described herein.

The components comprising the kits are typically provided in one or more containers. In certain preferred embodiments, the containers are sterile, or capable of being sterilized (e.g. tolerant of on site sterilization protocols).

The kits can be provided with instructional materials teaching users how to use the device of the kit. For example, the instructional materials can provide directions on utilizing the assay device to screen for modulators of ion channels.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

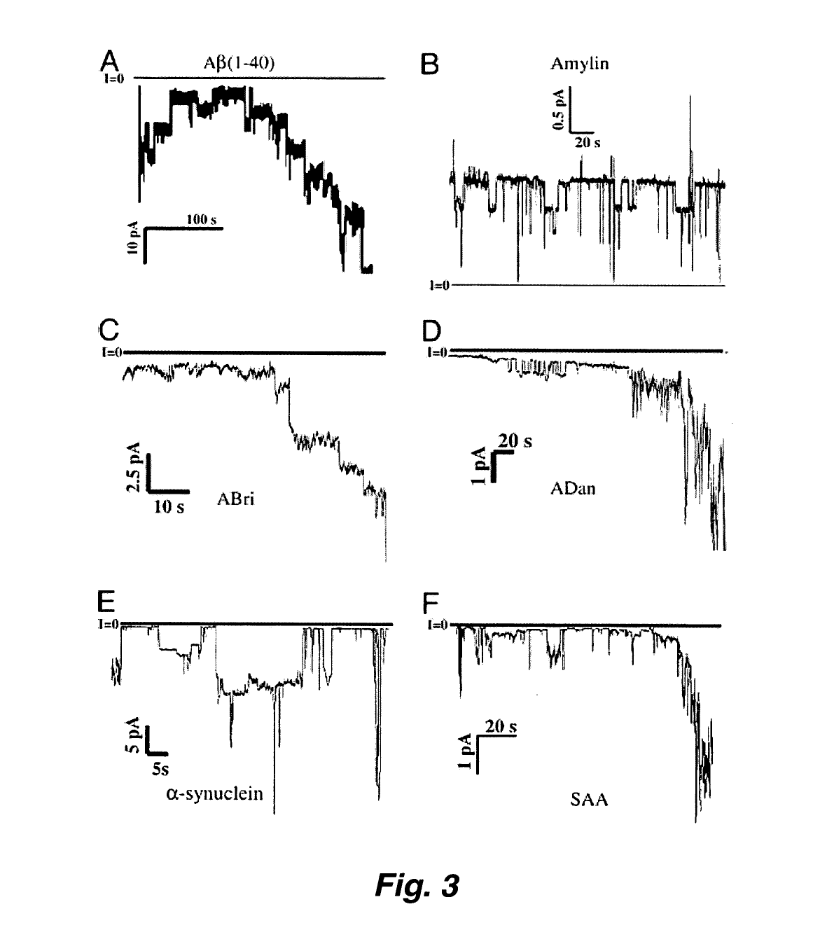

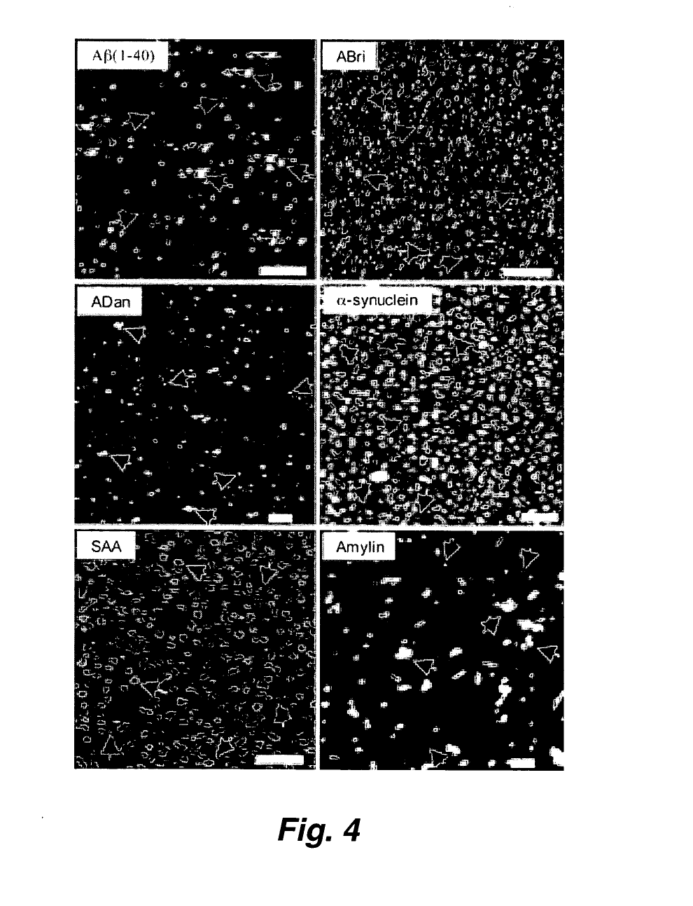

Using liposomes reconstituted with AbP channels in the membrane, one can screen for compounds that block AbP channels. FIG. 3 shows an experiment that Zn²⁺ and an anti-AbP antibody (3D6) blocks the channels formed by the 40 residue AbP₁₋₄₀ and inhibits the uptake of ⁴⁵CA²⁺ into the liposomes via the AbP channels. FIG. 4 shows a similar experiments, in which the channels formed by the 42-residue AbP₁₋₄₂ was blocked by and antibody, Zn²⁺, and Tris.

### Example 2

### Amyloid Ion Channels: A Common Structural Link for

### Protein-Misfolding Disease

Protein conformational diseases, including Alzheimer's, Huntington's, and Parkinson's result from protein misfolding giving a distinct fibrillar feature termed amyloid. Recent studies show that only the globular (not fibrillar) conformation of amyloid proteins is sufficient to induce cellular pathophysiology. However, the 3D structural conformations of these globular structures, a key missing link in designing effective prevention and treatment, remain undefined as yet. Using atomic force microscopy, circular dichroism, gel electrophoresis and electrophysiological recordings, we show here that an array of amyloid molecules, including Aβ(1-40), α-synuclein, ABri, ADan, Serum Amyloid A, and amylin undergo supramolecular conformational change. In reconstituted membranes, they form morphologically compatible ion-channel-like structures and elicit single ion channel currents. These ion channels would destabilize cellular ionic homeostasis and hence induce cell pathophysiology and degeneration in amyloid diseases.

Materials and Methods

1,2-Dioleoyl-sn-glycero-3-phosphocholine (DOPC) was purchased from Avanti Polar Lipids. Human α-synuclein recombinant protein (α-synuclein; molecular mass, 14.5 kDa) and human apo-serum amyloid A (SAA) were purchased from Alpha Diagnostics (San Antonio, Tex.) and PeproTech (Rocky Hill, N.J.), respectively. Aβ(1-40), amylin, ADan, and ABri were synthesized in the W. M. Keck Facility (Yale University) by N-t-butyloxycarbonyl chemistry and purified by reverse-phase HPLC. Hepes was purchased from Sigma, and 16.5% Tris-N-tris(hydroxymethyl)methylglycine (Tricine)˜SDS precast gel cassettes, SDS sample buffer, Tris-Tricine-SDS running buffer, and molecular mass standards were purchased from Bio-Rad. All solutions were prepared by using ultrapure water (resistivity˜18.2 M˜˜cm⁻¹) from Milli-Q from Millipore purification system.

CD Spectrometry.

Changes in the secondary structure were evaluated by monitoring the peptide species (typically 25-50 μg per 300 μl of 5 mM Tris, pH 7.4) spectrum in the far UV by using a J-720 spectropolarimeter (Jasco, Easton, Md.) at 1-nm intervals over the wavelength range 190-260 nm at 24° C. in a 0.1-cm path-length cell. Results are expressed in molar ellipticity (deg˜cm²˜mol⁻¹).

Polyacrylamide Gel Electrophoresis.

Freshly dissolved ABri, ADan, SAA, and α-synuclein were electrophoresed on a 16.5% Tris-Tricine polyacrylamide gel under reducing conditions without cross-linking, whereas amylin and Aβ(1-40) were electrophoresed under the same conditions but after covalent cross-linking using glutaraldehyde as described below. Extraction of peptide oligomers reconstituted in DOPC liposomes was performed by freeze-thawing of the lipid-peptide mixture followed by pelleting through centrifugation. The pellet was washed by using 10 mM Hepes solution (pH 7.4) and subsequently resuspended in 10 mM Hepes. The procedure was repeated three times to ensure that no unincorporated peptides were left in the mixture. Afterward, liposomes were dissolved in SDS sample buffer (200 mM Tris/HCl/2% SDS/40% glycerol/0.04% Coomassie blue G-250, pH 6.8). SDS sample buffer was added to peptides freshly dissolved in water. The peptides were separated by electrophoresis on 16.5% Tris˜Tricine˜SDS polyacrylamide gels (SDS˜PAGE). Molecular mass markers (from Bio-Rad) were run parallel to the samples. Peptides were fixed with 10% acetic acid and stained with Coomassie Blue G-250 (Invitrogen) or silver stain (Bio-Rad).

Cross-Linking of Aβ(1-40) and Amylin in DOPC Membrane and in Solution.

Without cross-linking, the amount of multimers in the gels for Aβ(1-40) and amylin was very small, most likely because they fall apart when heated up to 90° C. before running them through the gels. We cross-linked Aβ(1-40) and amylin oligomers reconstituted in DOPC membranes as described by Lin et al. (2001) FASEB J., 15: 2433-2444, by using 5011 of glutaraldehyde, added to 400 μl of DOPC/Aβ(1-40) and DOPC-amylin mixtures, to a final concentration of 12 mM glutaraldehyde. The reaction was stopped after 10 min for amylin and 20 min for Aβ(1-40), respectively, with 100 μl of Tris solution (1 M). Six microliters of glutaraldehyde was added to 24 μl (1 mg/ml) of Aβ(1-40) or amylin solutions in ultrapure water to a final concentration of 12 mM glutaraldehyde, followed by the addition of 20 μl of Tris/SDS/PAGE sample buffer after 10 min for amylin and 20 min for Aβ(1-40), respectively. Cross-linked products were solubilized in 2% SDS solution and analyzed by SDS/PAGE. For comparison, we also cross-linked nonmembrane-associated peptides.

Ion-Channel Current Measurements.

Planar phospholipid bilayer membranes were formed as described by Mirzabekov et al. (1999) Meth. Enzymol. 294: 61-74. A bubble of lipid dissolved in heptane was placed at the end of a small (100-300 μm) Teflon tube. Silver/silver chloride electrodes connected the aqueous components bounding the membrane to a voltage clamp. Ion-channel currents through the membrane were recorded by an Axopatch amplifier (Axon Instruments, Sunnyvale, Calif.). Data were filtered at 1 kHz and stored on VHS tape. Membrane capacitance and resistance were monitored continuously to ensure the formation and stability of reproducible membranes and the proper membrane thickness. Membranes that showed instability, abnormal capacitance, or abnormal resistance were not used. Control experiments with soluble proteins (e.g., BSA) showed that membranes did not interact with nonamyloid peptides. Peptide samples were introduced by perfusing the aqueous solution bounding one side of the membrane.

Sample Preparation for AFM Imaging.

Planar lipid bilayers were prepared by means of liposome fusion followed by rupture on the mica surface by procedure modified from Lin et al. (2001) FASEB J., 15: 2433-2444. Briefly, DOPC was dissolved in chloroform and dried under a flow of dry argon. DOPC pellet was vacuum-desiccated over-night and subsequently resuspended in 10 mM Hepes (pH 7.4) to a final concentration of 1 mg/ml. Lipids were hydrated for 1 h during which occasional vortexing was applied. Liposomes then were freeze-thawed and passed subsequently through a set of 400- and 200-nm pore size filters. Peptides were dissolved in ultrapure water and mixed with the DOPC liposomes at a 1:20 weight ratio. Lipid-protein mixture was bath-sonicated for 30 sec. Liposomes reconstituted with peptides then were deposited on freshly cleaved mica for 20 min and allowed to fuse and rupture upon contact with the mica surface forming planar lipid bilayers. The sample then washed, and no additional amyloids were added so that no unincorporated amyloids were left before imaging.

AFM Imaging and Image Analysis.

AFM images were acquired by using Nanoscope IIIa Multimode AFM with an Extender electronics module (Veeco, Santa Barbara, Calif.) as described in ref. 5. Oxide-sharpened silicon nitride cantilevers with a nominal spring constant of ˜0.06 N/m were used for most experiments. Imaging was carried out in both regular contact mode and in tapping mode (at oscillation frequencies between 9 and 15 kHz). Occasionally, higher-frequency resonance peaks (28-33 kHz) were used. The scan rates ranged between 1 and 12 Hz. All imaging was performed in 10 mM Hepes solution (pH 7.4) by using AFM liquid cell at room temperature. Through a continuous adjustment of the scanning parameters, it was ensured that imaging did not affect surface structure by routinely examining for damage by increasing the scan size at regular time intervals.

AFM images were processed and analyzed by using Veeco software. Some AFM images were low-pass filtered. Single ion channels images were passed through an additional low-pass Gaussian filter to reduce pixilation. Sizes of freshly dissolved peptide molecules as well as reconstituted channels in membrane were obtained by cross-sectional and bearing analyses software. The size of the structures observed in the cross-sections of height mode AFM images were measured at two-thirds of full height with respect to the substrate plane (mica surface for freshly dissolved nonmembrane-associated peptides; the bilayer membrane surface for amyloid channels) (Lin et al. (2001) FASEB J., 15: 2433-2444). Sizes and pore statistics for reconstituted channels were obtained from 50-200 channel-like features for each particle in amplitude-mode images. For the bilayers reconstituted with the peptide, often low gains in AFM imaging were required, rendering the amplitude image more reliable for analysis than the height images.

Results

Secondary Structure and Membrane-Induced Oligomerization.

The secondary structures of Aβ(1-40), α-synuclein, ABri, ADan, SAA, and amylin were evaluated by CD spectrometry. Various conformations were observed for the different amyloid peptides; Aβ(1-40) and α-synuclein showed predominantly unordered conformations, ADan and amylin were rich in β-structures, ABri was a mixture of β-sheet and random conformations, and SAA was basically α-helix (FIG. 1). The oligomeric nature of soluble globular amyloids before and after their reconstitution in bilayer membrane then was analyzed on SDS/PAGE. Freshly dissolved amyloid peptides appear predominantly monomeric with a strong band corresponding to their respective molecular masses (FIG. 2). Weaker bands corresponding to smaller amounts of dimers and higher-order oligomers are also present (FIG. 2). Conversely, amyloid peptides isolated after their reconstitution in liposomes appear as higher-order (trimers to octamers) oligomers at significantly higher concentration compared with their soluble counterparts (FIG. 1, left bands). The extent of membrane-induced oligomerization varied considerably among various peptides. Whereas amylin and Aβ(1-40) were predominantly trimeric to hexameric, α-synuclein and SAA were tetrameric to octameric, but ADan and ABri were only hexameric and tetrameric, respectively (FIG. 2).

These results indicate that in lipid bilayers, a significantly higher percentage of these amyloids are oligomers (trimers and larger), while a small percentage of monomers and dimers are also present. On the contrary, soluble amyloid peptides are primarily monomers or dimers with a small percentage of higher-order oligomeric complexes. In the lipidic environment, thus, amyloid peptides undergo conformational changes favoring larger oligomeric complexes, although some large oligomeric complexes of soluble peptides can still retain their structure when inserted in a lipidic membrane (Lin et al. (2001) FASEB J., 15: 2433-2444; Lashuel et al. (2002) Nature 418: 291). A presence of large oligomeric complexes in membrane suggests that they could form supramolecular structures.

Amyloid Peptides Induce Single Ion-Channel Currents When Reconstituted in Lipid Membrane.

We examined the activity of these oligomeric complexes in reconstituted bilayers by using a single-channel electrophysiological recording technique. All six amyloid peptides induced single-channel ion conductances when reconstituted in appropriate lipid bilayers. FIG. 3 shows an example of single-channel currents as a function of time across planar lipid bilayer membranes for each of these six peptides. With the exception of amylin (islet amyloid polypeptide, or IAPP), all amyloids formed channels with heterogeneous single-channel conductances, suggesting that several distinct oligomeric species formed channel structures (Lin et al. (2001) FASEB J., 15: 2433-2444). Single-channel recording could often be seen to merge into macroscopic conductances, implying that the former are responsible for the latter. One-sided addition of amyloid peptide to the solution bounding a bilayer membrane was sufficient to induce channel activity. Multiple sizes of single channels usually could be observed that depended on the peptide aggregation state. Channels were never observed in the absence of added peptide. Channel-forming activity could sometimes vary depending on the aggregation state of the peptide; e.g., disaggregation with DMSO followed by brief reaggregation in water often could enhance channel-forming ability.

A complete electrical characterization of amyloid channels was not the main focus of the work; rather, the goal was a confirmatory element to support the results that the 3D membrane structures of various amyloids that we report in this work indeed elicit ion-channel conductance and currents. Previous electrophysiological studies of amyloid peptides [Aβ(1-40) (Arispe et al. (1993) Proc. Natl. Acad. Sci. USA 90: 10573-10577), amylin (Mirzabekov et al. (1996) J. Biol. Chem. 271: 1988-1992), SAA (Hirakura et al. (2002) Amyloid 9: 13-23), and NAC (α-synuclein 60-95) (Kagan and Azimova (2003) Biohys. J. 84: 53A (abstract))] have characterized electrophysiological properties in detail that includes multiple conductances, ion selectivity, and roles of specific agonists, antagonists, and antibodies. In general, results obtained in the present work are consistent with earlier studies of these peptides. Previously unidentified ABri and ADan channel conductances are reported here: they both exhibit heterogeneous single-channel conductances and macroscopic conductance increases strikingly similar to those of other amyloid peptide channels (Kagan et al. (2004) J. Membr. Biol. 202: 1-10).

Amyloid Peptides Reconstituted in Bilayer Membrane Form Channel-Like Structures.

To understand the structural features of membrane-induced conformational changes, we used AFM to image 3D structures of these amyloids present in both native (soluble, non-membranous) form and when reconstituted in a lipid bilayer. AFM images of freshly dissolved peptides show globular features with average diameters of 1-10 nm (FIG. 4). Based on their size distributions in the AFM images and their comparison with images of other similar peptides (Lin et al. (2001) FASEB J., 15: 2433-2444; Zhu et al. (2000) FASEB J., 14: 1244-1254; Bhatia et al. (2000) FASEB J., 14: 1233-1243; Lin et al. (1999) Biochemistry 38: 11189-11196; Rhee et al. (1998) J. Biol. Chem. 273: 13379-13382; Lashuel et al. (2002) Nature 418: 291; Lashuel et al. (2003) J. Mol. Biol. 332: 795-808; Lashuel et al. (2002) J. Mol. Biol. 322: 1089-1102; Srinivasan et al. (2003) J. Mol. Biol. 333: 1003-1023; Ding et al. (2002) Biochemistry 41: 10209-10217), these globular structures appear to be mostly monomers and dimers, although higher-order oligomeric complexes cannot be ruled out. Significantly, unlike earlier reports indicating amyloids' tendency to form large fibrillar aggregates in solution, by using real-time AFM imaging we confirmed that these peptides retained their globular structure over a period of several (>4) hours with no significant change in the size distributions and without significant aggregation, even at physiologically high concentrations (>1 mg/ml) (data not shown). The complexity of their surface physiochemical properties was reflected in their adsorption to the mica substrate. It was difficult to adsorb soluble ADan and amylin on mica surface, which reflects the presence of very few monomeric or oligomeric complexes and mostly large aggregates in AFM images (FIG. 4). Conversely, ABri, α-synuclein, and SAA were highly attractive to the mica surface, which reflects the presence of both monomers as well as their high-density clusters.

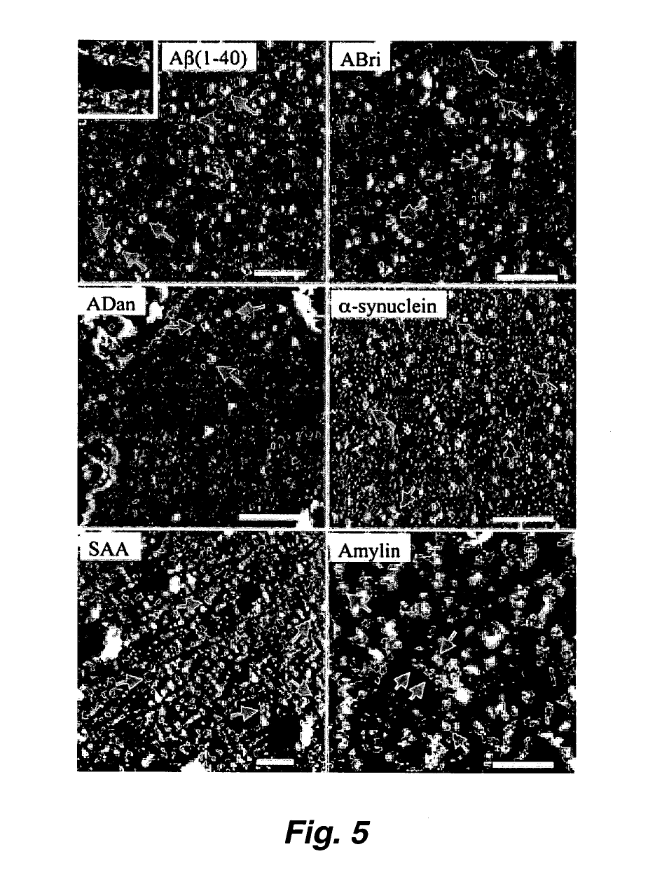

We then investigated the possibility that the observed ionic currents (FIG. 3) are indeed due to the pores formed by the globular oligomeric amyloids when they are reconstituted in bilayer membranes. We reasoned that freshly dissolved peptide molecules would adsorb on the liposome surface and, after undergoing partial folding, oligomerize in the lipid-bilayer membrane (Lin et al. (2001) FASEB J., 15: 2433-2444). Alternately, preformed oligomeric complexes with defined pores (Lashuel et al. (2002) Nature 418: 291; Lashuel et al. (2003) J. Mol. Biol. 332: 795-808; Lashuel et al. (2002) J. Mol. Biol. 322: 1089-1102) could insert directly in the membrane. Bilayers obtained from fusion of large liposomes were resolved in ˜75% of all reconstitution experiments. An example of a planar lipid-bilayer membrane reconstituted with Aβ(1-40) and immobilized on the mica surface is shown in FIG. 5 Inset. Consistent with earlier studies (Id.), the lipid bilayer shows flat patches, irregular in shape in the range of a few square micrometers in size and ˜5- to 5,5-nm thick [the thickness of a single bilayer membrane (Id.)]. When examined at higher resolution using AFM, the surface of the planar lipid-bilayer patch formed by only lipid vesicles without any amyloid peptides showed no distinguished features (data not shown). The average roughness of these surfaces varied by <0.1 nm.

Surfaces of lipid bilayers show that, once reconstituted in the lipid membranes, predominantly monomeric and dimeric globular peptides appear to coexist with stable higher-order multimers. At medium-resolution imaging (scan size 500-1,000 nm, 512-512 pixels), multimeric peptide complexes have disk-like shapes with an outer diameter of 8-12 nm and often contain a central pore-like concavity with a diameter of 1-2 nm (FIG. 5). These structures protrude ˜1 nm above the surrounding flat lipid-bilayer membrane. The presence of channel-like features varied among various amyloids, perhaps reflecting diversity in their interactions with lipids and their eventual stable insertion in the bilayer. On average, 66-75% of all reconstituted bilayers show globular multimeric complexes with the diameter of 10-12 nm, suggesting that they form supramolecular structures. In ˜20% of these structures, a central pore-like feature could be resolved, indicating the formation of channel-like structures. The presence of distinct central pore-like features could be an underestimation due to the AFM tip morphology (blunt tip) or local movement of subunits (due to the imaging in hydrated condition), or it could reflect the low rate of channel formation in reconstituted membrane. In most bilayer samples, larger structures also could be observed on or in the membrane but had no central pore-like feature.

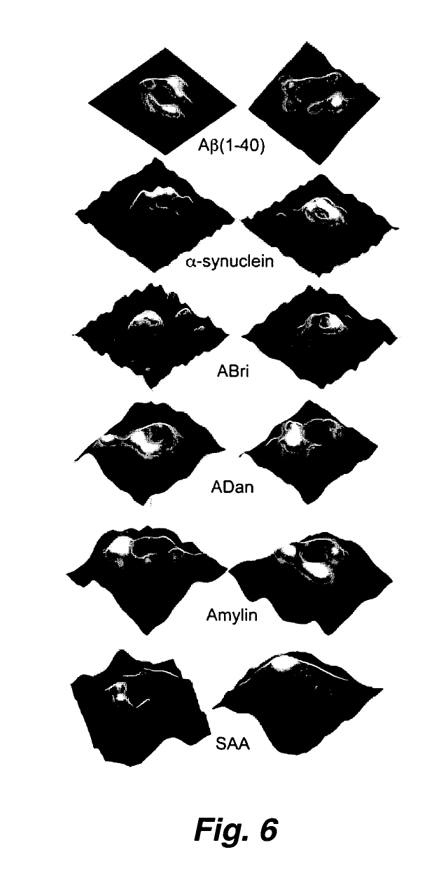

Upon closer examination of individual channel-like structures at higher resolution, several possible subunit arrangements were revealed: rectangular with four subunits, pentagonal with five subunits, hexagonal with six subunits, and octahedral with eight subunits (FIG. 6). Individual subunits' extramembranous protrusion varied by 0.2-0.5 nm. Aβ(1-40) and ADan were mainly four- and six-subunit structures. Up to eight subunits were observed only for SAA and α-synuclein. Pentamers were mainly observed for amylin. For ABri, resolution was only good enough to resolve substructures on few channels. The four-subunit channels, and in some cases the six-subunit channels, show an overall twofold rotational symmetry. It is possible that lower-order oligomers (e.g., dimers, trimers) could form higher-order complexes (tetramers, hexamers, etc.), although we did not ascertain their presence. Occasionally, subunits seem dislocated, breaking the symmetry of arrangement of the subunits. Pore sizes were smallest (˜1 nm) for ˜-synuclein and ABri and larger (˜2 nm) for Aβ(1-40), ADan, amylin and SAA. The differing multimeric structures and substructures of various amyloid peptides are consistent with data obtained from SDS/PAGE (FIG. 2) and size-exclusion chromatography (data not shown) and are consistent with the model of amyloid-membrane interactions (Temussi et al. (2003) EMBO J. 22: 355-361; Curtain et al. (2003) J. Biol. Chem. 278: 2977-2982; Mobley et al. (2004) Biophys. J. 86: 3585-3597), the 3D structure proposed by Durell et al. (1994) Biophys. J. 67: 2137-2145. The subunit variation is also consistent with the multiple electrical conductances observed in this work (FIG. 3) as well as others (Lin et al. (2001) FASEB J., 15: 2433-2444; Lin et al. (1999) Biochemistry 38: 11189-11196; Rhee et al. (1998) J. Biol. Chem. 273: 13379-13382; Kawahara et al. (2000) J. Biol. Chem. 275: 14077-14083; Arispe et al. (1993) Proc. Natl. Acad. Sci. USA 90: 10573-10577).

Discussion

Despite the substantial progress made in understanding the mechanisms underlying the formation of amyloid (amyloidosis) and its prevention, very little can be attributed to amyloidosis as the prime initiator of protein conformational diseases. Our present results show that soluble amyloid subunits, regardless of their initial secondary structure (FIG. 1), assume a supramolecular 3D structure when reconstituted in membrane bilayer (FIGS. 4-6). Conformations of soluble amyloids depend on several factors, including solvents, pH, and metals (Zn, Cu) (Curtain et al. (2003) J. Biol. Chem. 278: 2977-2982). In our study, we avoided any alcohol derivatives, samples were prevented from going through multiple phase transitions, and AFM imaging was performed in appropriate physiological conditions. Thus, the structures imaged in our study truly represent the supramolecular 3D features of globular amyloidogenic peptides. Globular amyloidogenic peptides have been reported to partition membrane in two phases (Green et al. (2004) J. Mol. Biol. 342: 877-887), and large globular complexes, usually termed as ADDLs, have induced nonspecific membrane disruption and leakiness (Kayed et al. (2004) J. Biol. Chem. 279: 46363-46366). In our earlier studies on calcium uptake in liposomes reconstituted with amyloids, we never observed any nonspecific calcium leakage (Lin et al. (1999) Biochemistry 38: 11189-11196; Rhee et al. (1998) J. Biol. Chem. 273: 13379-13382). Membrane-partition studies (16) had used preformed membranes adsorbed to a substrate before addition of peptides. Such supported bilayers have limited ability for refolding˜restructuring when incubated with amyloids and are inappropriate for ion channel reconstitutions; to our knowledge, in all published studies of ion-channel reconstitutions, either in artificial membranes (as in the present work) or in vivo in cell plasma membranes, bilayer membranes were accessible to peptides from the both sides.

The supramolecular 3D structure of reconstituted amyloid peptides in our work is similar to an ion channel (Lin et al. (2001) FASEB J., 15: 2433-2444; Lashuel et al. (2002) Nature 418: 291; Lashuel et al. (2003) J. Mol. Biol. 332: 795-808; Lashuel et al. (2002) J. Mol. Biol. 322: 1089-1102). We see a heterogeneous population of multimeric channels that vary for different amyloid peptides. Structural heterogeneity of amyloid channels [tetrameric to hexameric and higher-order structures (FIG. 6)] is consistent with the higher-order oligomeric transformation of monomeric and dimeric soluble peptides after their membrane insertion and correlates with the nature of the peptides (FIG. 2). This result is further supported by size-exclusion chromatography and spectral analysis of peptides isolated after insertion in the reconstituted membrane (data not shown). Moreover, such heterogeneity conforms to the original varying secondary structure, charge distribution, and tissue and disease specificity of the amyloid peptides that we have examined in this work. Structural and biochemical findings are supported by electrophysiological data that show heterogeneous single-channel conductances for these amyloids and are also consistent with previous studies that ion channels formed by various amyloid lengths exhibit multilevel channel conductances. These multilevel conductances could be due to the multiple conformational changes in the amyloid channel structure or could simply reflect the difference in the number of subunits that form a single channel (Kawahara et al. (2000) J. Biol. Chem. 275: 14077-14083; Arispe et al. (1993) Proc. Natl. Acad. Sci. USA 90: 10573-10577; Durell et al. (1994) Biophys. J. 67: 2137-2145; Hirakura et al. (1999) J. Neurosci. Res. 57: 458-466). Channel-forming activity also could vary with the nature of lipid and lipid mixtures (Arispe and Doh (2002) FASEB J. 16: 1526-1536; Lin and Kagan (2002) Peptides 23: 1215-1228). Nevertheless, our data show strongly that all these peptides induce ion-channel activity when reconstituted in bilayer membranes.

Amyloid ion-channels would provide the most direct pathway for inducing pathophysiological and degenerative effects when cells encounter amyloidogenic peptides; these channels would mediate specific ion transport (Lin et al. (2001) FASEB J., 15: 2433-2444; Lin et al. (1999) Biochemistry 38: 11189-11196; Rhee et al. (1998) J. Biol. Chem. 273: 13379-13382; Kawahara et al. (2000) J. Biol. Chem. 275: 14077-14083; Arispe et al. (1993) Proc. Natl. Acad. Sci. USA 90: 10573-10577; Hirakura et al. (2002) Amyloid 9: 13-23) and thus destabilize the cell ionic homeostasis. A loss of ionic homeostasis would increase the cell calcium to toxic levels, which is the common denominator for the early cellular event leading to pathophysiology and degeneration (Lin et al. (2001) FASEB J., 15: 2433-2444; Zhu et al. (2000) FASEB J., 14: 1244-1254; Bhatia et al. (2000) FASEB J., 14: 1233-1243; 19, 26). In vivo and in vitro studies have shown that amyloid molecules can form stable small oligomers at physiological concentrations (low nanomolar) as well as up to micromolar levels. The production, oligomerization, and degradation of these amyloids is a dynamic process. Under normal conditions, soluble amyloids are bound to various amyloid-binding proteins and are usually cleared from cerebrospinal fluid into the bloodstream, most likely via receptor transport mechanisms across the blood-brain barrier. In the diseased brain, the level of soluble amyloids is significantly elevated. This elevation could result in an excessive accumulation of amyloid in the cerebrospinal fluid and the formation of calcium-permeable amyloid channels in the cell plasma membrane. Continued accumulation of amyloid channels over an extended time period would eventually increase the disruptive level of cellular free calcium in a dose-dependent manner. With other cellular weaknesses as yet unidentified, toxic calcium level would lead to cellular dysfunction and degeneration. The cellular toxicity data from several recent studies support such a scenario.

In summary, our data provide clear evidence that various amyloid molecules indeed form pore-like structures and elicit channel activity in membrane. Our results provide the structural identity of globular amyloid complexes that would induce pathophysiological cellular activity and degeneration resulting from protein misfolding; amyloid ion channels would allow ionic exchange across the plasma membrane and thus disrupt the cellular ionic homeostasis. Overwhelming electrophysiological evidence suggests that such ionic exchange ultimately leads to cellular calcium loading, the common denominator of the amyloidogenic cellular pathophysiology and degeneration.

### Example 3

### An on-Chip Detection System for Ion Channel Activity: AFM Imaging and Electrical Current Recording Through Bilayers Supported Over Microfabricated Silicon Chip Nanopores

In this example, we describe a silicon chip based supported bilayer system to detect the presence of ion channels and their electrical conductance in lipid bilayers. Nanopores were produced in microfabricated silicon membranes by electron beam lithography as well as by using a finely focused ion beam. Thermal oxide was used to shrink pore sizes, if necessary and to create an insulating surface. The chips with well defined pores were easily mounted on a double chamber plastic cells recording system allowing for controlling the buffer conditions both above and below the window. The double chamber system allowed using an AFM tip as one electrode and inserting a platinum wire as the second electrode under the membrane window, in order to measure conductance across lipid bilayers that are suspended over the pores. Atomic force imaging and stiffness measurement and electrical capacitance measurement indicate the feasibility of supporting lipid bilayer over well defined nanopores. On-line addition of gramicidin, an ion channel forming peptide resulted in characteristic ionic conductance measured using IV curve measurements. This system is ideally suited for direct 3D structure-function study of channel conformation.

Here we report on the fabrication of nanopores in silicon membranes that can be used for supported bilayer study with two fluid compartments one each below and above the channels, respectively. These silicon chips with nanopores support reconstituted lipid bilayers. The lipid bilayers are stiff enough to allowing their imaging with AFM. Using conducting AFM tips, electrical current was recorded for ion channels formed in the lipid bilayer and over the chip nanopores by online addition of Gramicidin, an ion channel forming peptide.

Results and Discussion

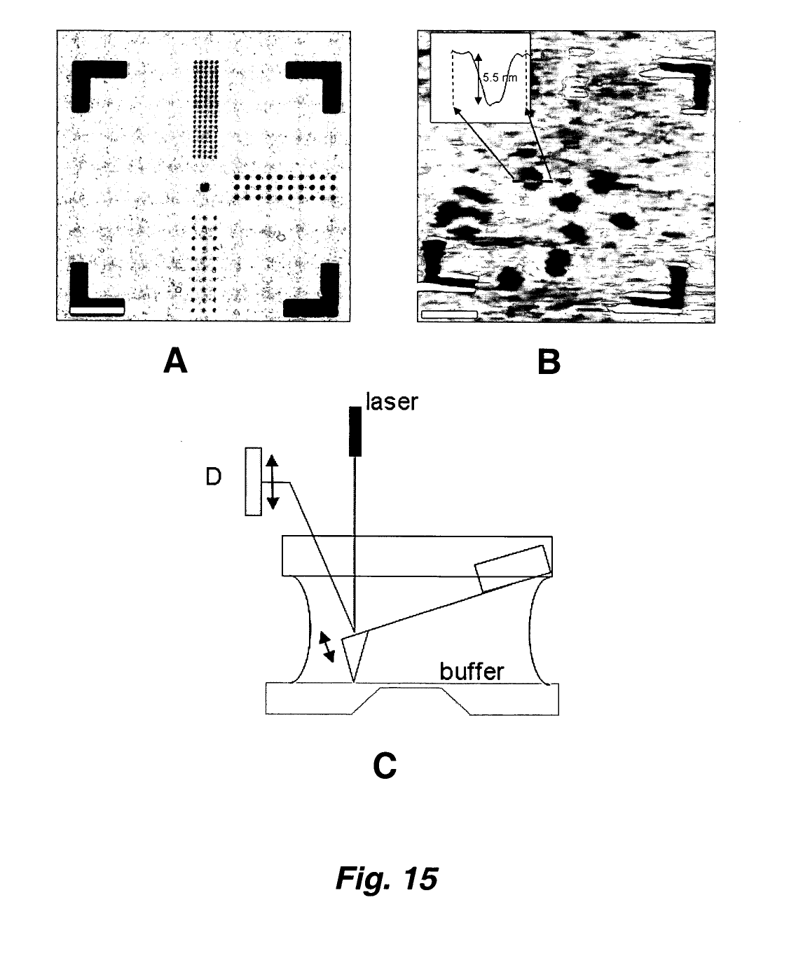

AFM imaging of initial test pores produced by electron beam lithography shows pores with a diameter ranging from 50 nm to 200 nm or more. An example of such nanopores is shown in FIG. 15. The large markers were used to find the patterns of interest by an optical microscope that is integrated with the AFM. After deposition of a lipid bilayer over the chip, the pores in the chip are covered by the bilayer. The larger corner markers are still visible since the bilayers collapse over such large openings, allowing for easy navigation, and as reference to where the nano pores are situated under the bilayer (FIG. 15).

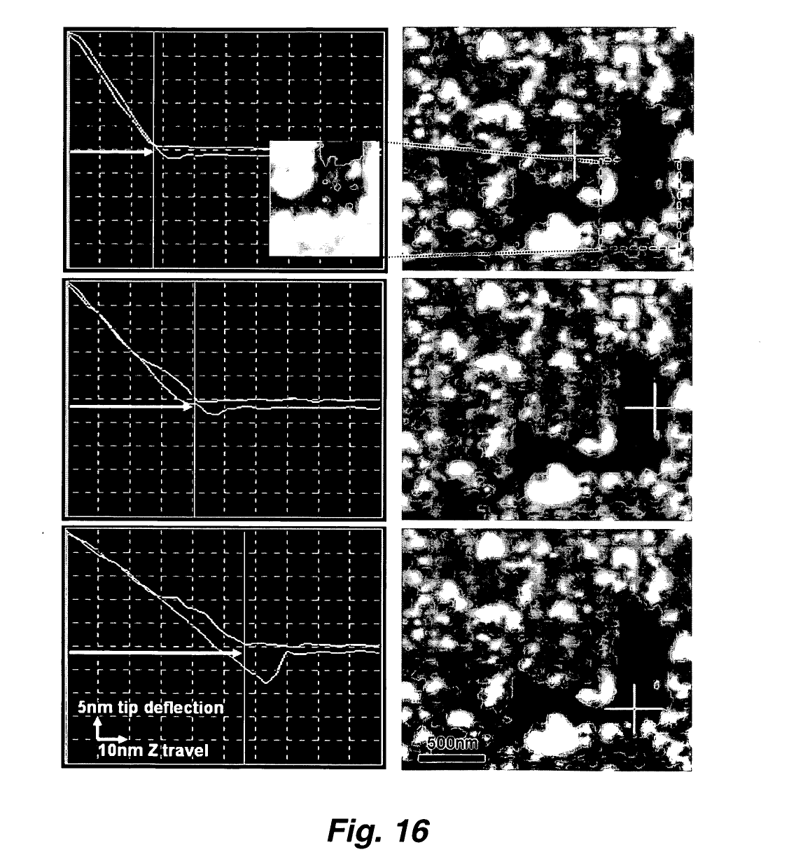

FIG. 15, panel B shows a bilayer supported over the chip. The bilayer covers the pores in the chip and also reveals several holes in the bilayer. The AFM cross-sectional height measurements along those holes indicate the hole depth of ˜5.5 nm, the nominal thickness of a common lipid bilayer (Lin et al. (2001) FASEB J., 15: 2433-2444). The bilayers are sometimes strong enough to span even the 500 nm wide gap of the alignment marks. FIG. 16 shows a situation where a bilayer is suspended over one part of an alignment mark, but ruptures in the other part. Most likely the bilayer was ruptured during its deposition and not during AFM imaging, since the remaining suspended part of the bilayer was stable for several AFM scans.

AFM force curve measurements show the stiffness of a bilayer on the silicon nitride membrane (FIG. 16 top), of a bilayer broken along the edge of an alignment mark (FIG. 16 middle), and of a bilayer suspended over the opening (FIG. 16 bottom). The increased amount of z-travel necessary to obtain the same cantilever deflection when the AFM tip is over the suspended bilayer vs for the bilayer on the silicon nitride substrate indicates that the bilayer is indeed suspended over the alignment mark hole. The indication of a slightly softer surface at the edge of the hole is most likely caused by the AFM tip contacting the edge of the bilayer from the side. The inset in FIG. 16 shows a suspended bilayer in more detail, the imaging force (˜1.5 nN) is low enough to have a stable membrane for repetitive imaging.

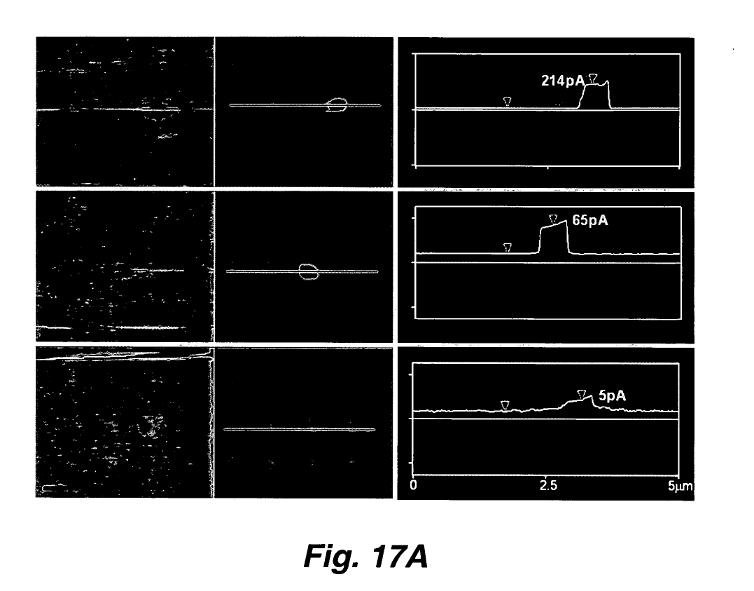

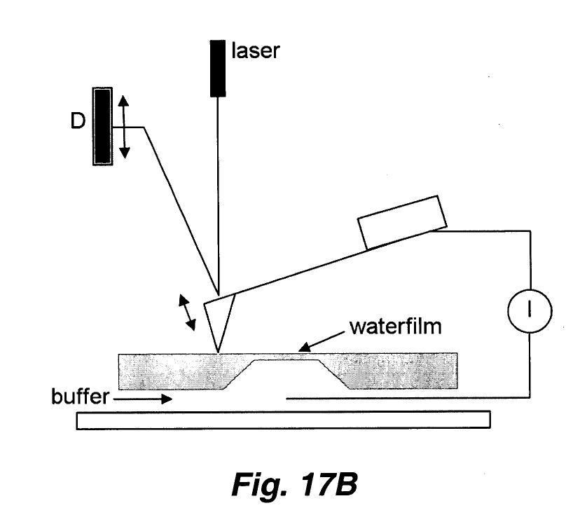

For measuring current through the nanopores in the chip, pores produced by FIB in silicon windows were used. The pores in the chip were insulated and shrunk in size by thermal oxide deposition using plasma enhanced CVD. They do not contain the large alignment markers that would give rise to leakage current as is the case in our electron beam lithography produced pores, and only one pore is milled in each die. Furthermore, the FIB milling process is a direct one step process, eliminating the need for photo- or electron beam resist materials potentially contaminating the sample. In order to measure electrical conductance through a single pore, 135 mM KCl solution was used under the nitride membrane in the cell containing the bottom electrode, and the platinum coated AFM tip was used as the second electrode measuring current while imaging the structure simultaneously. In order to measure local (not the bulk) conductance through the complete cantilever holder, no buffer solution was used on top of the membrane, only humid air was gently flowed over the surface to maintain a water meniscus between the tip and sample to get only conductance through the tip apex and not the rest of the cantilever holder assembly. Results of simultaneous imaging and conductance measurements are shown in FIG. 17.

The pore is imaged repeatedly using different bias voltages applied to the bottom electrode located under the silicon nitride membrane in the 135 mM KCl solution. The height image does not show the pore depth and size properly, and the pores appear shallow. This is caused by tip induced broadening due to the large tip radius of the platinum coated tips. The current images show bright spots where there is a current flowing between bottom electrode and tip. The ‘current spots’ are larger than the pore, indicating that the water layer due to humidity near the pores is conductive enough to allow current to flow thus current is not completely limited to the on-pore area exclusively. Increasing the bias voltage applied to the bottom electrode under the pore chip from −0.5 Volt to −1.0 Volt and subsequently to −2.0 Volts, resulted in an increase in the current respectively from 5 to 65 pA, and from 65 to 214 pA, respectively (FIG. 17). This indicates the ability to detect ion channel conductance of 10 pS or lower with these tip electrodes and of large support pores. For small pores as of many ion channels and for localized current flow, the sensitivity of the conducting tips could be sufficient to measure single ion channel conductance.

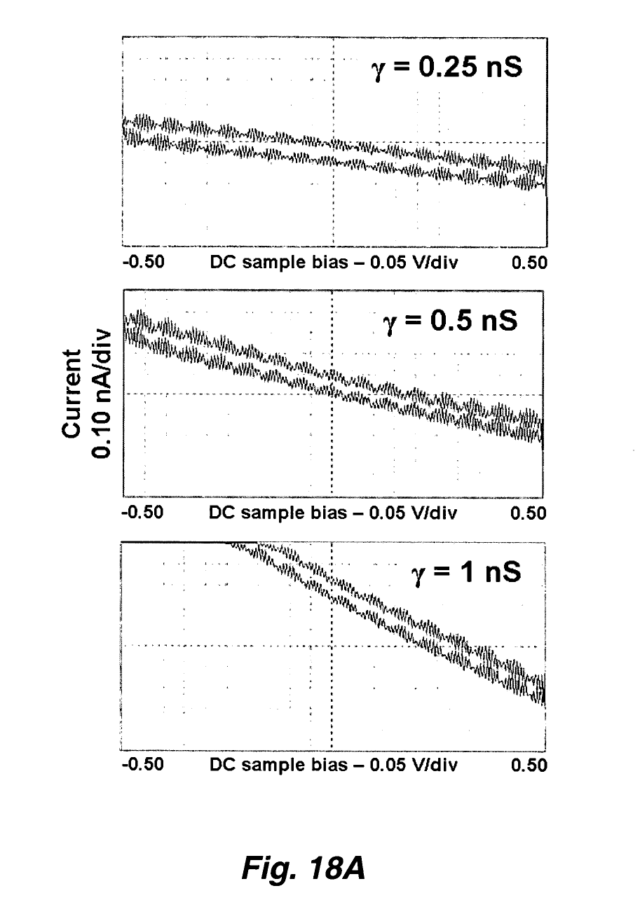

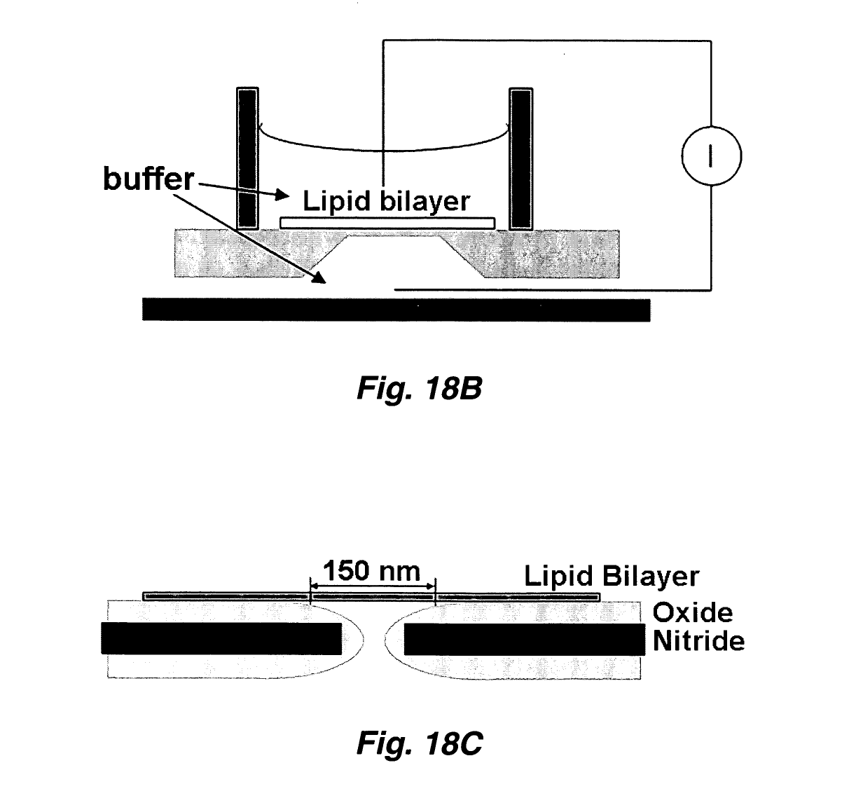

In order to study the sealing effect of the bilayer over the nanopores in the chip, lipid vesicles were deposited over to chips. After 30 minutes for a bilayer to form from vesicular fusion, the excess unadsorbed lipids and vesicles were rinsed away from the surface. Bulk electrical conductance across the supported nanopore chip and the overlying adsorbed lipid bilayer was measured using a drop of 135 mM KCl solution on top of the chip in which the AFM tip holder assembly was submerged and 135 mM KCl under the nitride membrane. The IV curve measured was near flat and indicates a conductance across the bilayer of 0.025 nS. To check for the possibility of using the nanopore chip to measure ion channel conductance, Gramicidin (a known ion channel forming peptide) was added to the solution at a concentration of 0.2 mg/ml. The effect of Gramicidin on conductance is shown in FIG. 18, while applying a 1V bias to the bottom electrode under the silicon nitride membrane.

Conductance increased within seconds after addition of gramicidin. After 4 minutes the increase in conductance stabilized at roughly 1 nS (FIG. 18). Since the Gramicidin is dissolved in the same water based KCl solution as present above and below the bilayer, an increase in the conductance is due to ion channel formation, and not due to a solvent induced leaky membrane. No current was measured in absence of other proteins and there was no non-specific leaky current otherwise. This shows that gramicidin forms ion channels in the lipid bilayer in random positions, some of which will coincide with the location of one of the nanopores through the underlying silicon nitride membrane. The conductance of a gramicidin ion channel is usually small, varies considerably, is dependent upon the solvent, and influenced considerably by the nature of the detergents. Assuming a nominal conductance of approx 10 pS, we estimate the number of functional gramicidin ion channels overlaying the nanopore in the supported chip to be approx 100. No effort was made to measure single channel conductance. Moreover, no effort was made to image individual ion channels. For simultaneous high resolution imaging and conductance measurements, one can utilize a sharp tip (e.g., a nanotube) with capability to measure local conductance in fluid through the tip apex only. We have developed such a nanotube tip (Chen et al. (2006) Appl. Phys. Letts., 88 (153102) and using such a tip it is it is possible to make simultaneous conductance/imaging studies at the single ion channel level.

In summary, we report the design of a nanopore chip suitable for use as a support for lipid bilayer membranes with or without embedded channels and receptors where in both sides of the extramembranous portions of these channels and receptors are accessible for on-line pharmacological and biochemical perturbations. The system allows for simultaneous AFM imaging and electrical recording and thus opening the possibility to study the direct structure-function relation of ion channels with high resolution: it will be possible for on-line gating of ion channels and imaging their structural features in open and closed states while recording ionic current passing through the channels. With a further development of AFM tip technology that will allow for AFM tips that are conducting only at the final apex without the risk of contamination (as in wax coated tips), this nanopore chip allows for simultaneous molecular resolution imaging and single channel electrical recording

Experimental Design

Two approaches were used to produce nanopores. As an initial test, pores were produced in 200 nm thick silicon nitride windows (SPI Supplies, West Chester Pa.) using electron beam lithography combining arrays of pores of varying sizes with large markers to be easily located by optical microscopy for easier navigation of the AFM tip to the pores. Patterning was performed using a Jeol JBX 5DII system (Jeol, Peabody Mass.) with a LaB6 electron source at 50 kV acceleration voltage, 50-100 pA current, and ZEP520 as high resolution resist. After developing the patterns in 100% amyl acetate, a Bosch Deep Reactive Ion Etch was used to etch the pores through the silicon nitride windows. After final strip and cleaning, image of pores were obtained by SEM as well as AFM (Veeco Metrology, Santa Barbara, Calif.).

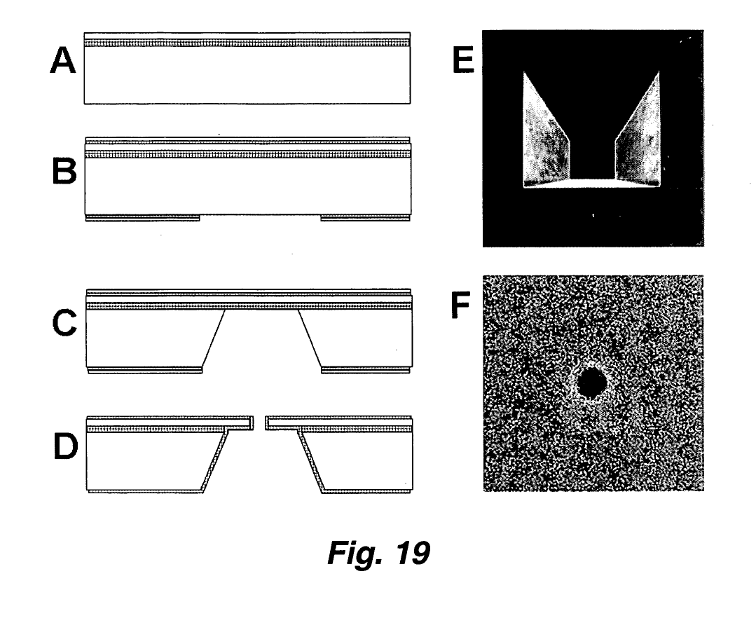

Secondly, silicon membranes were used. They were microfabricated using Silicon-on-Insulator (SOI) wafers. As shown in FIG. 19, panel A the SOI wafer consists of a device layer, buried oxide layer and handle wafer of thicknesses 0.34 μm, 0.3 μm and 300 μm, respectively. A thermal oxide of 300 Å thickness followed by a 1000 Å low stress LPCVD silicon nitride was deposited as masking layers for etching silicon in a KOH solution. A photolithography step was performed to open a window in the silicon nitride and oxide layers to expose the silicon wafer for etching as shown in FIG. 19, panel B. Wafers were then etched using 33% aqueous KOH solution at 70° C. to produce arrays of dies 7 mm×7 mm in size, each having a pyramidal shaped opening of 200 μm×200 μm in the center. Etching was stopped at the buried oxide layer thus leaving an oxide and Si membrane window. The wafer after this step is shown in FIG. 19, panel C, and an SEM image of the wafer backside with the pyramidal shaped opening is shown in FIG. 19, panel E. The LPCVD silicon nitride layer was removed in hot phosphoric acid. The oxide layers were stripped in buffered HF leaving a silicon membrane. The dies were processed using a focused ion beam (FIB International), creating one nanopore, 70-150 nm in diameter, through the Si membrane in each die. A 70 nm diameter pore is shown in FIG. 19, panel F. In order to provide electrical insulation, thermal oxide of various thicknesses was grown on both the top and bottom sides of the die. The final structure is shown in FIG. 19, panel D.

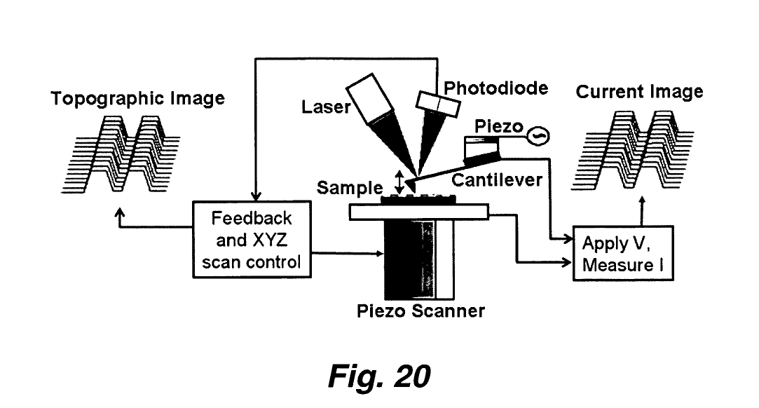

For all samples, nano pore membranes were mounted on plastic liquid cells to exchange fluid above or below the membrane. IV curves were obtained in 135 mM KCl solution. Current was measured using the conductive AFM setup with the cantilevered tip holder as an electrode on the top and a reference electrode under the pore-chip (FIG. 20). In absence of an insulating coat, when buffer was present on both sides of the chip, current is measured through the complete tip holder clip. To image current only through the AFM tip apex, conductance was measured with 135 mM KCl solution only under the pore chip and a flow of humid air was maintained over the top surface. This set up also ensured that, by allowing only a local water meniscus between tip and the sample, only the local conductance through each pore was measured.

AFM was also used to image pores after deposition of lipid vesicles (DOPC) prepared by previously described method (Lin et al. (1999) Biochemistry, 38: 11189-11196). Briefly, vesicles were formed by drying 1 mg of DOPC lipid in a glass tube, kept in a desiccator overnight, and rehydrated in buffer with occasional sonication. For vesicle deposition a droplet (50 micro liter, 1 mg/ml) of vesicles was placed on the pore chip, allowed to adsorb for 30 minutes, and rinsed with buffer. After bilayer deposition and before IV measurements, ionic strength was brought back to 135 mM KCl both on top as well as below the pore chip to keep the bilayer hydrated properly, and current was measured through the tip holder. Once a proper seal was achieved by the bilayer, Gramicidin, an ion channel forming peptide (dissolved in milliQ water with 135 mM KCl), was added to the buffer solution, and the current was measured as a function of time while gramicidin interacted with the bilayer.

### Example 4

### Extremely Sharp Carbon Nanocone Probes for Atomic Force Microscopy Imaging

The key component of atomic force microscopy (AFM) is the probe tip, as the resolution and reliability of AFM imaging is determined by its sharpness, shape, and the nature of materials. Standard commercial probes made of silicon or silicon nitride have tips of a pyramid shape, that do not allow easy access to narrow or deep structural features, and generally have a relatively blunt tip radius on the order of 10 nm. The high-aspect-ratio geometry and excellent mechanical strength of carbon nanotubes (CNTs) offer advantages for imaging as an AFM tip. Due to their excellent physical and chemical properties (Dresselhaus et al., editors Carbon Nanotubes: Synthesis, Structure, Properties, and Applications, Springer, Berlin, 2001; Bower et al. (2002) Appl. Phys. Lett. 80: 3820-2002; Fennimore et al. (2003) Nature, 424: 408). CNTs have been attached onto pyramid tips by various approaches (Dai et al. (1996) Nature, 384: 147; Nishijima et al. (1999) Appl. Phys. Lett. 74: 4061; Stevens et al. (2000) Appl. Phys. Lett. 77: 3453; Hall et al. (2003) Appl. Phys. Lett. 82: 2506; Tang et al. (2005) Nano Lett. 5: 11) as well as directly grown using thermal chemical vapor deposition (CVD) (Hafner et al. (1999) Nature, 398: 761; Cheung et al. (2000) Appl. Phys. Lett. 76: 3136; Yenilmez et al. (2002) Appl. Phys. Lett. 80: 2225). The attachment methods are manual and time consuming, and often result in nonreproducible CNT con-figuration and placement. While the thermal CVD approach can potentially lead to the wafer-scale production of AFM tips, the number, orientation, and length of CNTs are difficult to control.

An important aspect to consider in utilizing CNT probes is that the single-walled nanotube probes with a desirable small diameter tend to exhibit an inherent thermal vibration problem if the length is made reasonably long, and hence they cannot be used to trace deep structural profiles. On the other hand, multiwalled nanotubes such as those synthesized in dc plasma enhanced CVD (dc-PECVD) (Merkulov et al. (2002) Appl. Phys. Lett. 80: 4816; Chen et al. (2004) Appl. Phys. Lett. 85: 5373; AuBuchon et al. (2004) Nano Lett. 4: 1781; Chhowalla et al. (2001) J. Appl. Phys. 90: 5308) have a larger diameter in the regime of 20-100 nm and hence exhibit improved mechanical and thermal stability, but the catalyst particle at the nanotube probe tip (or the natural dome structure in a nanotube grown by a base growth mechanism) has a finite radius of curvature, that limits the AFM resolution.

Recently, two approaches have been employed to fabricate multiwalled nanotube probes on tipless cantilevers by dc-PECVD (Ye et al. (2004) Nano Lett. 4: 1301; Cui et al. (2004) Nano Lett. 4: 2157). These approaches, however, require some what complicated, multiple patterning steps. The catalyst dots in both approaches are patterned by the lift-off of the spin-coated polymethyl methacrylate (PMMA) layer following typical electron-(e-beam) lithography. A reliable and uniform spin coating of a resist layer generally requires a relatively large area, and is difficult to achieve for a tipless cantilever, which has a narrow and elongated geometry. In one of these reports, (Ye et al. (2004) Nano Lett. 4: 1301) patterned catalyst dots were formed before the fabrication of the cantilevers, but the catalyst had to be protected by the PECVD-deposited Si₃N₄ layer in order for the catalyst dots to survive and keep catalytic activity throughout the subsequent microfabrication steps. In the other report, (Cui et al. (2004) Nano Lett. 4: 2157) the e-beam lithography steps had to be used twice to pattern a catalyst dot on the commercial tipless cantilever in order to remove the extra Ni catalyst on the cantilever. The probe tip radii reported are also relatively large.

In this example, we fabricated high-aspect-ratio carbon nanocone (CNC) probes with very sharp tips on tipless cantilevers by employing a resist-free e-beam induced deposition (EBID) of carbon masks combined with electric-field-controlled CVD growth. A high resolution AFM imaging of nanoscale features and deep grooves are demonstrated using CNC probes.

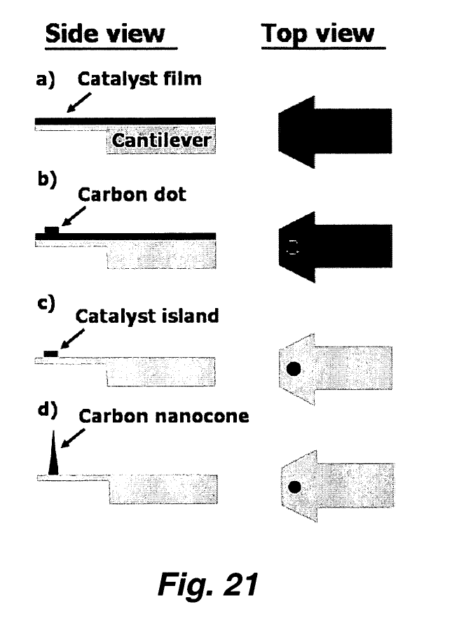

The fabrication process for a CNC probe is schematically illustrated in FIG. 21. First, the top surface of the cantilever (NSC/tipless, MikroMasch, USA) was coated with a ˜10 nm thick Ni film by e-beam evaporation. For the EBID of carbon dots, a JEOL IC845 scanning electron microscope (SEM) with the NPGS software (J. C. Nabity lithography system) was used. The acceleration voltage was 30 kV, and the beam current was 50 pA. The carbon dot deposition time was varied between 8 and 30 s depending on the intended size of the dot. No special carbonaceous precursor molecules were introduced as the residual carbon-containing molecules naturally presenting in the chamber were sufficient for the EBID processing to form amorphous carbon dots on the cantilever surface. A single carbon dot with a selected diameter of ˜300 nm was deposited near the front end edge of the cantilever by the EBID as illustrated in FIG. 21, panel B. The carbon dot serves as a convenient etch mask for chemical etching. The Ni film was then etched away by using a mixture of [H₃PO₄][HNO₃][CH₃COOH][H₂O]=1:1:1:2 except the portion underneath the mask. The removal of the carbon dot mask after the catalyst patterning was performed with an oxygen reactive ion etch (RIE) for 1 min, which exposed the Ni island as illustrated in FIG. 21, panel C. The cantilever with the Ni island was then transferred to the dc-PECVD system for subsequent growth of the CNC, FIG. 21, panel D. The growth of the CNC probe was carried out at 700° C. for 10-20 min using a mixture of NH₃ and C₂H₂ gas (ratio 4:1) at 3 mTorr pressure. An applied electric field was utilized to guide the growth of the nanocone along the desired direction.

The EBID of the carbon nanodots is a simple writing technique to directly fabricate nanoscale patterns on the substrate bypassing the use of any e-beam resist layer related steps (Broers et al. (1976) Appl. Phys. Lett. 29: 596). The carbon deposition is caused by the dissociation of the volatile molecules adsorbed on the substrate into a nonvolatile deposit via a high-energy focused electron. Compared with the typical e-beam lithography approach of preparing a single dot pattern on a small cantilever, the EBID process can more accurately pattern the catalytic island at the desired position via in situ control under high magnification of ×10 000 or higher in the SEM.

While the use of the EBID carbon patterns have been demonstrated as dry etching masks, (Broers et al. (1976) Appl. Phys. Lett. 29: 596) there has been no report for their use as wet etching masks to the best of our knowledge. We investigated the chemical etchability of the carbon dots in various acids and other chemicals such as HCl, HF, HNO₃, H₂O₂, and acetone, and found that the carbon dots were very stable and remained adherent on the substrate after immersing into these chemicals. The carbon dots have a unique advantage in that while they are resistant to chemical etching, they are easily removable by oxygen RIE. We find that the oxygen RIE process does not affect the Ni film and reduces its catalytic activity for CNT/CNC nucleation and growth. Experimental studies on CNC growth indicate that the diameter of the 10 nm thick Ni catalyst island should be kept smaller than ˜300 nm to avoid the undesirable nucleation and growth of multiple CNCs. Our carbon island chemical etch mask technique is generally useful for creating a pattern on any small samples such as a prefabricated tipless cantilever, on which the resist layer cannot be uniformly coated for reliable lithography.

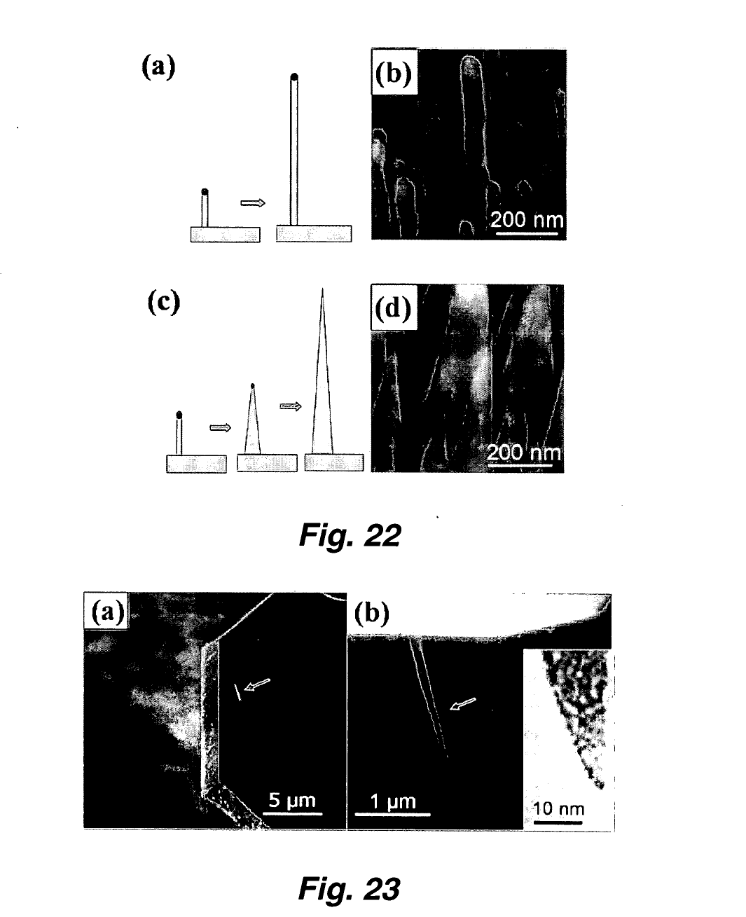

By adjusting the applied bias in the dc-PECVD system, the morphology of CNTs can be controlled. At a low applied voltage of 450 V, the size of the catalyst particles does not change during growth and the resultant CNTs are equidiameter nanotubes as shown in FIG. 22, panels A and B. When the applied voltage is increased, e.g., 550 V, the diameter of the catalyst particle on the tip can be gradually reduced due to a plasma etching ˜sputtering˜ effect, as indicated in FIG. 22, panels C and D. The gradually diminishing catalyst size causes the nanotube diameter to change with the growth time, resulting in a nanocone configuration and the eventual complete elimination of the catalyst particle at the CNC tip, which is the key mechanism to obtain a very sharp tip.

FIG. 23, panels A and B show the SEM images of our CNC probe (marked by an arrow) grown on a tipless cantilever. In FIG. 23, panel A, a single CNC probe grown near the edge of a tipless cantilever is shown. FIG. 23, panel B, a higher magnification SEM micrograph, shows a CNC with ˜2.5 μm height, 200 nm base diameters, and a cone angle <5°. The inset in FIG. 23, panel B is an example transmission electron microscopy (TEM) image of a CNC tip, which shows the tip radius of the curvature of only a few nanometers. The microstructure of the nanocone appears to be a mixture of crystalline and amorphous phases in general agreement with previous work (Chen et al. (2004) Appl. Phys. Lett. 85: 5373; Wang et al. (2005) Relat. Mater. 14: 907). It should be noted that the CNC probe in FIG. 23, panel B, is made intentionally tilted by manipulating the electric field direction during CVD growth. Such a tilted probe is desirable as it compensates the operation tilt angle of the AFM cantilever so that the probe itself is close to being vertical for stable imaging. The tilt angle of the CNC probe is ˜130 with respect to the normal direction of the cantilever surface.

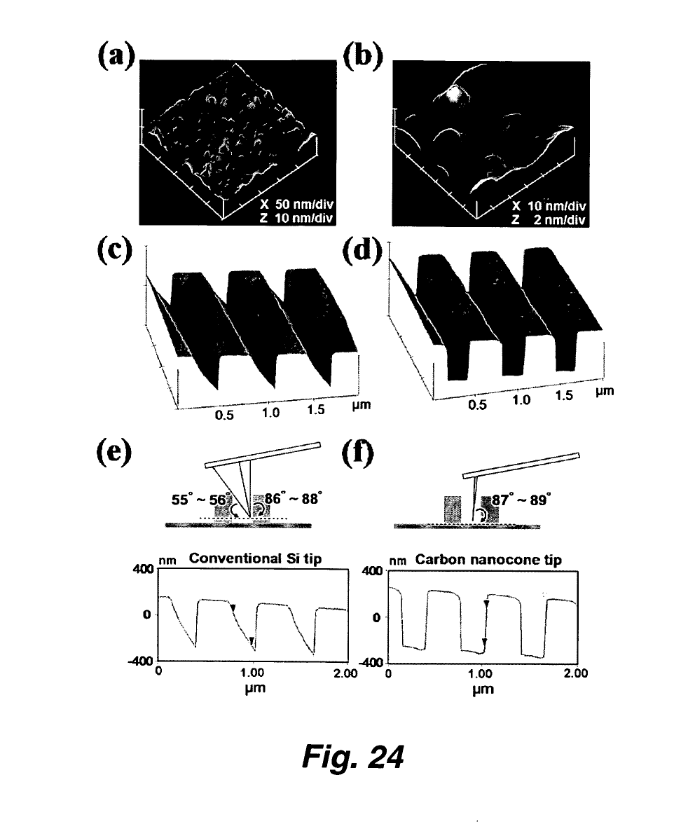

The performance of the CNC probe was evaluated in the tapping mode using a Dimension 3100 AFM with a Nano-scope IIIa controller (VEECO Instruments) for imaging in air. The surface of a copper film (˜300 nm thick) sputter deposited on the Si surface was imaged by using our CNC probe, as shown in FIG. 24, panels A-C. These images clearly show a well-defined and rounded grain structure, even for the grain size of ˜5 nm or smaller. The sharper grain boundaries and image quality were well revealed due to the sharpness of the CNC tip.

To demonstrate the advantage of the high aspect ratio of a CNC tip, a 300 nm line/space, 500 nm deep PMMA pattern was evaluated and the imaging performance of the conventional Si tip versus our CNC tip was compared. The image acquired with a Si probe, FIG. 24, panel C, shows a misleading image pattern due to the angle of the pyramid. The CNC probe, on the other hand, reveals the true geometry of the pattern including the configuration of the vertical walls implying that the CNF probe is able to trace the contour of the deep profile pattern, as presented in FIG. 24, panel D. From FIG. 24, panel E, the imaged right sidewall slope of 55°-56° and a left sidewall slope of 86°-88° of the pattern actually matches those of the Si pyramid shape itself. From a calculation of geometry, the Si pyramid tip cannot possibly reach the bottom of the PMMA pattern. The experimental results also confirmed this. As shown in FIG. 24, panel F, the sidewall slopes of the same pattern acquired with the CNC probe are about 87°-89°. These data demonstrate that the CNC probe is strong enough to trace the abrupt step structure without breakage.

In order to evaluate the mechanical durability and adhesion strength of the CNC probe, the probe was operated on a continuous scan mode on Au or Cu film samples for as long as 8 h. The lateral resolution of the obtained AFM image was not noticeably changed as compared to the initially scanned image at time zero (data not shown).

In summary, the fabrication of a sharp and high-aspect-ratio carbon nanocone probe that possesses desirable thermal stability and mechanical toughness has been demonstrated using resist-free patterning of catalyst nanodots and electric field guided CVD growth. The catalyst particle on the nano-cone tip was completely removed via time-dependent size reduction, thus leading to an extremely sharp tip, that can be used for AFM imaging and deep profile analysis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A device for screening for molecules that alter ion channel activity, said device comprising a lipid bilayer attached to a solid support, wherein said lipid bilayer contains one or more ion channel proteins.

2. The device of claim 1, wherein said solid support comprises one or more nanopores.

3. The device of claim 2, wherein said nanopores range in size from about 10 to about 400 nm in diameter.

4. The device of claim 2, wherein said nanopores range in size from about 20 to about 200 nm in diameter.

5. The device of claim 2, wherein said nanopores range in size from about 50 to about 100 nm in diameter.

6. The device of claim 2, wherein said nanopores penetrate through a surface having a thickness of 400 nm or less.

7. The device of claim 2, wherein said nanopores penetrate through a surface having a thickness of 200 nm or less.

8. The device of claim 2, wherein said nanopores are formed in a membrane.

9. The device of claim 2, wherein said nanopores are formed in a silicon wafer.

10. The device of claim 1, wherein said device provides a fluid reservoir on one side of said lipid bilayer.

11. The device of claim 1, wherein said device provides a fluid reservoir at each side of said lipid bilayer.

12. The device of claim 1, wherein said one or more ion channel proteins are selected from a group consisting of a calcium channel, a sodium channel, a potassium channel, a chloride channel, and a magnesium channel.

13. The device of claim 1, wherein said one or more ion channel proteins are amyloid proteins.

14. The device of claim 1, wherein said one or more ion channel proteins are AbP channel proteins.

15. The device of claim 1, wherein said device further comprises a means for detecting alteration of channel conformation in response to contact with a compound.

16. The device of claim 15, wherein said means comprises an AFM or an SPM.

17. The device of claim 15, wherein said means provides a measure of channel conductivity.

18. The device of claim 15, wherein said means provides both a measure of channel conductivity and channel protein conformation.

19. The device of claim 18, wherein said means provides a measure of channel conductivity and additionally comprises an AFM or an SPM.

20. The device of claim 1, wherein said device comprises a plurality of different channels.

21. The device of claim 20, wherein said device comprises at least 10, 20, 50, or 100 different channels.

22. The device of claim 20, wherein a plurality of said channels are each aligned with a pore in said solid support.

23. A method of screening a test agent for the ability to alter conductivity or conformation of an AbP channel, said method comprising:
contacting a device according to claims 1 through 21 with a test agent; and
detecting a change in conformation and/or conductivity of a channel in response to the contact with said test agent.

24. The method of claim 23, wherein said change in conformation is measured using AFM or SPM.

25. The method of claim 23, wherein said change in conductivity is measured using an AFM or SPM tip as an electrode.

26. The method of claim 23, wherein a change in conformation and a change in conductivity are measured simultaneously.

27. An AFM or SPM having an integrated carbon nanotube cantilever and tip.

28. A method of screening test agents for the ability to alter pore conformation or conductance by amyloid proteins, said method comprising:
providing a lipid bilayer comprising a pore comprising one or more amyloid proteins;
contacting said lipid bilayer with a test agent; and
detecting a change in the conformation and/or conductance of said pore, wherein a change in conformation and/or conductance indicates that said test agent alters pore conformation or conductance.

29. A carbon nanocone, said nanocone comprising, wherein said nanocone comprises a high-aspect ratio carbon nanotube structure substantially lacking a catalyst at the tip.

30. The nanocone of claim 29, wherein said nanocone has a cone angle of less than about 10 degrees.

31. The nanocone of claim 29, wherein said nanocone has a cone angle of less than about 5 degrees.

32. The nanocone of claim 29, wherein said nanocone has an aspect ratio (height:base) of at least about 10:1.

33. The nanocone of claim 29, wherein said nanocone has an aspect ratio (height:base) of at least about 12:1.

34. The nanocone of claim 29, wherein said nanocone has a tip radius of less than about 10 nm.

35. The nanocone of claim 29, wherein said nanocone has a tip radius of less than about 5 nm.

36. The nanocone of claim 29, wherein said nanocone has a tip radius of less than about 3 nm.

37. A method of fabricating a nanocone, said method comprising a resist-free e-beam induced deposition (EBID) of carbon masks combined with electric-field-controlled CVD growth.

38. The method of claim 37, wherein said method comprises utilizing EBID carbon patterns as dry etching masks.
